(19) United States
(12) Patent Application Publication
(10) Pub. No.: US 2018/0070819 A1
(43) Pub. Date: Mar. 15, 2018

(54) CAMERA SYSTEM, FEEDING SYSTEM, IMAGING METHOD, AND IMAGING DEVICE

(71) Applicant: Panasonic Intellectual Property Management Co., Ltd., Osaka (JP)

(72) Inventors: KATSUHIRO KANAMORI, Nara (JP); JUN OZAWA, Nara (JP)

(21) Appl. No.: 15/820,481

(22) Filed: Nov. 22, 2017

Related U.S. Application Data
(63) Continuation of application No. PCT/JP2016/004094, filed on Sep. 8, 2016.

(30) Foreign Application Priority Data
Nov. 5, 2015 (JP) 2015-217964

(51) Int. Cl. A61B 3/14; A61B 3/11; A61B 3/12; A01K 29/00; A01K 5/02; A61B 3/00

(52) U.S. Cl. CPC A61B 3/14; A61B 3/112; A61B 3/12; A01K 11/004; A01K 5/02; A61B 3/0025; A61B 2503/40; A01K 29/005

(57) ABSTRACT

A camera system that captures images of the eyeballs of an animal is provided with: a first illumination device that illuminates an eyeball of the animal; a fundus imaging camera that captures a fundus image of the eyeball illuminated by the first illumination device; a second illumination device that illuminates an eyeball of the animal at the same timing as the first illumination device; a pupil imaging camera that captures a pupil image of the eyeball illuminated by the second illumination device; and an output circuit that outputs the fundus image as identification information of the animal, and outputs the pupil image as biological information of the animal corresponding to that identification information.

### BACKGROUND

### 1. Technical Field

The present disclosure relates to a camera system and the like with which an image of an eyeball of an animal is captured.

### 2. Description of the Related Art

A camera system that photographs an eyeball of an animal such as a cow has been proposed in the past (for example, Japanese Patent No. 5201628). In the camera system of Japanese Patent No. 5201628, light is illuminated onto a pupil of an animal, the intensity of reflected light that is reflected by that pupil is measured using a camera, and the intensity of that reflected light is converted into a vitamin A blood concentration of the animal. This vitamin A blood concentration is used as biological information of that animal.

### SUMMARY

However, in the aforementioned camera system of Japanese Patent No. 5201628, there is a problem in that it is not possible for the biological information of the animal to be acquired while appropriately identifying that individual animal.

A non-limiting and exemplary aspect of the present disclosure is able to acquire the biological information of an animal while appropriately identifying that individual animal.

In one general aspect, the techniques disclosed here feature a camera system that captures images of the eyeballs of an animal, provided with: a first illumination device that illuminates an eyeball of the animal; a fundus imaging camera that captures a fundus image of the eyeball illuminated by the first illumination device; a second illumination device that illuminates an eyeball of the animal at the same timing as the first illumination device; a pupil imaging camera that captures a pupil image of the eyeball illuminated by the second illumination device; and an output circuit that outputs the fundus image as identification information of the animal, and outputs the pupil image as biological information of the animal corresponding to the identification information.

It should be noted that general or specific aspects hereof may be realized by a device, a system, a method, an integrated circuit, a computer program, or a computer-readable recording medium, and may be realized by an arbitrary combination of a device, a system, a method, an integrated circuit, a computer program, and a recording medium. A computer-readable recording medium includes a nonvolatile recording medium such as a compact disc read-only memory (CD-ROM).

According to the present disclosure, the biological information of an animal can be acquired while that individual animal is appropriately identified. Additional benefits and advantages of the aspects of the present disclosure will become apparent from the present specification and drawings. The benefits and/or advantages may be individually provided by the various aspects and features disclosed in the present specification and drawings, and need not all be necessary in order to obtain one or more of the same.

### BRIEF DESCRIPTION OF THE DRAWINGS

FIG. 1 is a drawing depicting a camera system in embodiment 1;

FIG. 2 is a drawing depicting an example of the arrangement positions of a fundus imaging camera and a pupil imaging camera in embodiment 1;

FIG. 3 is a flowchart depicting an imaging method for capturing images of the eyeballs of an animal in embodiment 1;

FIG. 4 is a drawing depicting a camera system in embodiment 2;

FIG. 5A is a drawing depicting a usage example of the camera system in embodiment 2;

FIG. 5B is a drawing depicting a usage example of the camera system in embodiment 2;

FIG. 6 is a drawing depicting an example of a configuration of a first illumination device and a fundus imaging camera in embodiment 2;

FIG. 7 is a drawing depicting an example of the first illumination device and the fundus imaging camera seen from an optical axis direction in embodiment 2;

FIG. 8 is a drawing depicting an example of a configuration of a second illumination device and a pupil imaging camera in embodiment 2;

FIG. 9 is a drawing depicting an example of the second illumination device and the pupil imaging camera seen from an optical axis direction in embodiment 2;

FIG. 10 is a drawing depicting an example of the arrangement positions of the fundus imaging camera and the pupil imaging camera in embodiment 2;

FIG. 11 is a drawing depicting timings for illumination performed by the first illumination device and imaging performed by the fundus imaging camera in embodiment 2;

FIG. 12 is a drawing depicting an example of a histogram of a clear infrared fundus image in embodiment 2;

FIG. 13A is a drawing depicting a relationship between the line of sight of an eyeball and the first illumination device and fundus imaging camera in embodiment 2;

FIG. 13B is a drawing depicting a relationship between the line of sight of an eyeball and the first illumination device and fundus imaging camera in embodiment 2;

FIG. 14 is a drawing depicting timings for illumination performed by the second illumination device and imaging performed by the pupil imaging camera in embodiment 2;

FIG. 15 is a block diagram of an analysis unit in embodiment 2;

FIG. 16 is a drawing for describing a method for measuring the velocity of pupil constriction due to light in embodiment 2;

FIG. 17A is an explanatory diagram of a light emission method for the second illumination device in embodiment 2;

FIG. 17B is an explanatory diagram of a light emission method for the second illumination device in embodiment 2;

FIG. 18 is a drawing for describing another method for measuring the velocity of pupil constriction due to light in embodiment 2;

FIG. 19 is a drawing depicting timings for illumination performed by the first illumination device and the second illumination device in embodiment 2;

FIG. 20A is a drawing depicting image information displayed on a mobile terminal in embodiment 2;

FIG. 20B is a drawing depicting estimate information displayed on the mobile terminal in embodiment 2;

FIG. 21 is a flowchart depicting an imaging method for capturing images of the eyeballs of an animal in embodiment 2;

FIG. 22 is a drawing depicting timings for illumination performed by a second illumination device and imaging performed by a pupil imaging camera in embodiment 3;

FIG. 23 is a drawing depicting an analysis unit and a control unit in embodiment 4;

FIG. 24 is a flowchart depicting an example of a control method for a camera system in embodiment 4;

FIG. 25 is a flowchart depicting another example of a control method for the camera system in embodiment 4;

FIG. 26A is a flowchart depicting an example of the control of a second illumination device and a pupil imaging camera performed by the control unit in embodiment 4;

FIG. 26B is a flowchart depicting another example of the control of the second illumination device and the pupil imaging camera performed by the control unit in embodiment 4;

FIG. 26C is a flowchart depicting another example of the control of the second illumination device and the pupil imaging camera performed by the control unit in embodiment 4;

FIG. 27 is a drawing depicting a camera system in embodiment 5;

FIG. 28 is a drawing in which the camera system in embodiment 5 is seen from above;

FIG. 29 is a drawing depicting an example of a configuration of a feeding system in embodiment 6;

FIG. 30 is a block diagram of an analysis unit in embodiment 6;

FIG. 31A is a drawing in which an animal eye imaging device according to embodiment 7 is seen from the side;

FIG. 31B is a drawing in which the animal eye imaging device according to embodiment 7 is seen from the side;

FIG. 32 is a drawing in which the animal eye imaging device according to embodiment 7 is seen from the front;

FIG. 33 is a drawing in which the animal eye imaging device according to embodiment 7 is seen from above;

FIG. 34 is a drawing describing a configuration of a white light source-equipped color camera in embodiment 7;

FIG. 35A is a drawing depicting details of illumination in embodiment 7;

FIG. 35B is a drawing depicting details of illumination in embodiment 7;

FIG. 35C is a drawing depicting details of an imaging element in embodiment 7;

FIG. 36A is a drawing depicting another configuration for polarized illumination in embodiment 7;

FIG. 36B is a drawing depicting another configuration for polarized illumination in embodiment 7;

FIG. 37A is a drawing depicting a spectral distribution of a light source in embodiment 7;

FIG. 37B is a drawing depicting a spectral distribution of imaging in embodiment 7;

FIG. 38 is a drawing describing a principle whereby color imaging is performed at a timing at which the line of sight of an eyeball of a cow is directly facing an imaging optical axis in embodiment 7;

FIG. 39 is a flowchart describing an algorithm in embodiment 7;

FIG. 40A is a drawing depicting a pupil image of an eyeball of a cow in embodiment 7;

FIG. 40B is a drawing depicting a pupil image of an eyeball of a cow in embodiment 7;

FIG. 41 is a drawing depicting a principle for separating two regions in embodiment 7;

FIG. 42 is a drawing depicting an experiment for separating a tapetum region using a simulated retina model in embodiment 7;

FIG. 43A is a drawing depicting a polarization imaging device of embodiment 8;

FIG. 43B is a drawing depicting a planar structure of a monochrome polarization image sensor in embodiment 8;

FIG. 44A is a drawing depicting a cross-sectional structure of an objective lens opening and a color filter region in embodiment 8;

FIG. 44B is a drawing depicting a color filter arrangement in embodiment 8;

FIG. 45 is a drawing describing processing for pixel selection and reintegration in which a color polarized image is generated from imaging results obtained using a microlens array-type of color image sensor in embodiment 8;

FIG. 46A is a drawing depicting a polarization imaging device of embodiment 9;

FIG. 46B is a drawing depicting a planar structure of a color imaging element in embodiment 9;

FIG. 47A is a drawing depicting a cross-sectional structure of polarizing filter regions of an opening in embodiment 9;

FIG. 47B is a drawing depicting a planar structure of the polarizing filter regions in embodiment 9;

FIG. 48 is a drawing describing pixel selection and reintegration processing in which a color polarized image is generated from imaging results obtained using a microlens array-type of color image sensor in embodiment 9;

FIG. 49A is a drawing depicting a polarization imaging device in embodiment 10;

FIG. 49B is a drawing depicting the polarization axes of polarizing filters corresponding to openings of four multi-objective lenses in embodiment 10; and

FIG. 50 is a drawing describing pixel selection processing in which a polarized image is generated from imaging results obtained by using a multi-lens color camera in embodiment 10.

### DETAILED DESCRIPTION

### (Findings Forming the Basis for the Present Disclosure)

Conventionally, vitamin A is maintained in a deficient state in the fattening period for cows in order for the meat quality of beef cattle to have a highly marbled state (marbled meat). However, severe illnesses such as blindness are caused when there is an excessive deficiency in vitamin A, and therefore measuring the vitamin A blood concentration of beef cattle is an important examination. In the past, this measurement has been carried out by collecting blood from cows; however, there have been problems in that the stress placed on the cows is regarded as an issue from the viewpoint of animal welfare and the examination time is long. Thus, technology has been developed in which an image of the pupil of an eyeball of a cow is captured in a non-contact manner, and the vitamin A blood concentration is determined from the pupil color by means of image processing. In an eyeball of a cow, there is a layer called the tapetum lucidum (hereinafter, the tapetum) extending across a region that is behind the retina and is approximately half the size of the retina. This tapetum has the role of increasing eye sensitivity by reflecting incident light in such a way that at night the incident light transmits through the retina twice. When an image of a pupil of a cow is captured using illumination and a camera, intense reflected light of the blue-green color of the tapetum is observed.

In Japanese Patent No. 5201628, an analysis is carried out based on the empirical fact that, in a cow having a vitamin A deficiency, the retina atrophies and the pupil color of the eye therefore becomes increasingly blue as the color of the blue tapetum is reflected. That is, reflected light having a wavelength of 400 nm to 600 nm reflected by the pupil is measured, and a regression analysis between that intensity and the vitamin blood concentration is carried out.

Furthermore, in Shuqing HAN, Naoshi KONDO, Yuichi OGAWA, Shoichi MANO, Yoshie TAKAO, Shinya TANIGAWA, Moriyuki FUKUSHIMA, Osamu WATANABE, Namiko KOHAMA, Hyeon Tae KIM, Tateshi FUJIURA, “Estimation of Serum Vitamin A Level by Color Change of Pupil in Japanese Black Cattle”, an analysis is carried out using the finding that the red component increases and the saturation decreases from among the color components of the pupil of an eyeball of a cow having a vitamin A deficiency. That is, the color of the pupil is observed using a color camera that has a light shielding tube and a white ring illumination device and that is capable of imaging practically in close contact with an eyeball of a cow, and a regression analysis between that red component and the vitamin A blood concentration is carried out.

Furthermore, in Tatsuya MORISAKO, Tateshi FUJIURA, Shinya TANIGAWA, Shuqing HAN, Naoshi KONDO, Yuichi OGAWA, Moriyuki FUKUSHIMA, Namiko KOHAMA, “Development of Individual Automatic Pupil Image Measurement Device for Beef Cattle”, The Japanese Society of Agricultural Machinery, June 2013, No. 114, p. 67, a non-contact imaging device is described as an imaging system that is installed in an actual cattle barn. Unnecessary stress is placed on a cow when a camera is brought into contact with an eyeball of the cow, and therefore, to avoid this, a device is described that automatically captures an image of the pupil of an eye of a cow in a non-contact manner at a timing at which the cow drinks water at night.

Furthermore, in Shuqing HAN, Naoshi KONDO, Tateshi FUJIURA, Yuichi OGAWA, Yoshie TAKAO, Shinya TANIGAWA, Moriyuki FUKUSHIMA, Osamu WATANABE, Namiko KOHAMA, “Machine Vision Based Prediction of Serum Vitamin A Level in Japanese Black Cattle by Pupillary Light Reflex Analysis”, a method is described in which the velocity of pupil constriction, due to a pupillary reflex in the case where light is radiated onto a pupil, and a start timing are observed by means of video image processing of the pupil, and a vitamin blood concentration is estimated therefrom.

In order for images of the pupils of both eyes of a cow to be captured in a non-contact manner, in the system disclosed in Tatsuya MORISAKO, Tateshi FUJIURA, Shinya TANIGAWA, Shuqing HAN, Naoshi KONDO, Yuichi OGAWA, Moriyuki FUKUSHIMA, Namiko KOHAMA, “Development of Individual Automatic Pupil Image Measurement Device for Beef Cattle”, The Japanese Society of Agricultural Machinery, June 2013, No. 114, p. 67, a color imaging device having a white ring illumination device is installed to the left and right of a water drinking station for the cow. On the basis of information from a distance sensor, white light is radiated and color imaging is carried out at a timing at which the cow is close to the optimum position. Here, it is necessary to identify the cow to which the automatically captured image corresponds from among a plurality of cows inside a cow pen, as in Tatsuya MORISAKO, Tateshi FUJIURA, Shinya TANIGAWA, Shuqing HAN, Naoshi KONDO, Yuichi OGAWA, Moriyuki FUKUSHIMA, Namiko KOHAMA, “Development of Individual Automatic Pupil Image Measurement Device for Beef Cattle”, The Japanese Society of Agricultural Machinery, June 2013, No. 114, p. 67. Presently, the individual identification (hereinafter, also referred to as individual authentication) of a cow is carried out by means of radio frequency identification (RFID) or photographing the number of an ear tag of the cow using an individual authentication camera installed at a position above the head of the cow. However, besides the drawbacks that RFID tags and ear tags are easily lost and can also be altered, pain is caused to the animal upon attachment.

A method in which a fundus image is acquired and the blood vessel pattern on the retina is used, as in Japanese Patent No. 4291514, is known as a method for individually identifying a cow in a non-contact manner in such a way that individual authentication accuracy is high and also pain is not caused to the cow. However, because the illumination and focus are different in devices that capture images of pupils and devices that capture images of the fundus, it has been difficult for images of a pupil and a fundus to captured at the same time using one device.

The present disclosure solves the aforementioned problems, and provides a camera system with which the biological information of an animal can be acquired while that individual animal is appropriately identified. Specifically, a camera system is provided with which a lesion examination for a vitamin A deficiency and the individual identification of a cow can be performed at the same time with images of a pupil and a fundus being captured at the same time in a non-contact manner.

A camera system according to an aspect of the present disclosure is a camera system that captures images of the eyeballs of an animal, provided with: a first illumination device that illuminates an eyeball of the animal; a fundus imaging camera that captures a fundus image of the eyeball illuminated by the first illumination device; a second illumination device that illuminates an eyeball of the animal at the same timing as the first illumination device; a pupil imaging camera that captures a pupil image of the eyeball illuminated by the second illumination device; and an output circuit that outputs the fundus image as identification information of the animal, and outputs the pupil image as biological information of the animal corresponding to the identification information.

Thus, by using two cameras, a fundus image constituting identification information of an animal and a pupil image constituting biological information of that animal can be acquired at the same time. As a result, the identification of the animal and the acquisition of biological information can be carried out quickly. Furthermore, in the camera system according to the aspect of the present disclosure, a second illumination device illuminates an eyeball of the animal at the same timing as the first illumination device. Consequently, a pupil image of the eyeball illuminated by the second illumination device can be appropriately captured even if pupil constriction is about to start or even if the animal is about to run away due to the eyeball being illuminated by the first illumination device in order to capture the fundus image, for example. Consequently, in the camera system according to the aspect of the present disclosure, the biological information of an animal can be acquired while that individual animal is appropriately identified.

Furthermore, the first illumination device may be an infrared illumination device or a white illumination device, and the second illumination device may be a white illumination device.

Thus, an infrared image or a color image in which a clear blood vessel pattern is depicted to a degree enabling the animal to be identified can be acquired as a fundus image, and a color image enabling the pupil color to be specified can be acquired as a pupil image. That is, the individual identification of the animal and the acquisition of biological information can be carried out appropriately.

Furthermore, an infrared illumination device and a line of sight detection unit that detects the line of sight of the animal may be additionally provided, the fundus imaging camera capturing a fundus image for detecting the line of sight of the eyeball illuminated by the infrared illumination device, the line of sight detection unit detecting the line of sight of the animal using the fundus image for detecting the line of sight, the first illumination device and the second illumination device illuminating the eyeballs, based on the detected line of sight of the animal, and the fundus imaging camera capturing the fundus image of the eyeball, and the pupil imaging camera capturing the pupil image of the eyeball. For example, the first illumination device and the second illumination device may illuminate the eyeballs when the detected line of sight of the animal is the same as the imaging optical axis of the fundus imaging camera.

Thus, because the eyeballs are illuminated based on the line of sight of the animal, when the line of sight of that animal is directed toward the fundus imaging camera 104, namely when the pupil of the eyeball is directly facing the fundus imaging camera, that eyeball is illuminated by the first illumination device, and a fundus image of the illuminated eyeball can be captured. Consequently, a fundus image having a clearer blood vessel patter depicted therein can be acquired, and highly accurate identification information can be acquired. Furthermore, the second illumination device illuminates the eyeball of the animal at the same timing as the first illumination device, and the pupil imaging camera captures a pupil image of that illuminated eyeball. Consequently, it is possible to suppress the line of sight of the animal deviating greatly from the pupil imaging camera, namely the pupil of the eyeball not directly facing the pupil imaging camera, when the pupil image is captured. As a result, a clear pupil image can be acquired, and highly accurate biological information can be acquired.

Furthermore, the second illumination device may emit light within 0.3 sec from the point in time at which the first illumination device emitted light.

Thus, the biological information of an animal can be acquired while that individual animal is appropriately identified, with reduced effect from pupil constriction or the animal running away due to the eyeballs being illuminated.

Furthermore, a measurement unit that measures the pupil constriction velocity of the animal may be additionally provided, the second illumination device once again illuminating the eyeball of the animal, within 0.3 sec from the point in time of having emitted light at the same timing as the first illumination device, the pupil imaging camera capturing a plurality of pupil images in accordance with the illumination performed by the second illumination device, and the measurement unit measuring the pupil constriction velocity of the animal using the plurality of pupil images.

Thus, a highly accurate pupil constriction velocity of the animal can be measured, with reduced effect from pupil constriction or the animal running away due to the eyeballs being illuminated.

Furthermore, when an angle formed by the illumination optical axis of the first illumination device and the imaging optical axis of the fundus imaging camera is θ1, and an angle formed by the illumination optical axis of the second illumination device and the imaging optical axis of the pupil imaging camera is θ2, the condition θ1≦θ2 may be satisfied.

Thus, the fundus imaging camera is able to observe the retina from the pupil in a state in which the light that is output from the first illumination device has reached the retina behind the pupil. As a result, the blood vessel pattern on the retina illuminated by the first illumination device can be appropriately captured as a clear fundus image.

Furthermore, the fundus imaging camera may have a first objective lens, the pupil imaging camera may have a second objective lens, and, when the distance between the first objective lens and the position of the surface of an eyeball of the animal is L1, and the distance between the second objective lens and the position of the surface of an eyeball of the animal is L2, the condition L1<L2 may be satisfied.

The position of the fundus of an animal is located further to the rear than the pupil surface, and therefore, because L1<L2, images of the fundus and the pupil can be captured at approximately the same viewing angle.

Furthermore, an identification unit that identifies the individual animal using the fundus image may be additionally provided, and the animal may not be illuminated by the second illumination device when the identification unit is not able to identify the individual animal.

Thus, a pupil image being acquired as biological information can be prevented until it is not possible to identify the animal, and wasteful processing and the accumulation of information can be eliminated.

Furthermore, a determination unit that determines whether or not the fundus image includes a lesion may be additionally provided, and the animal may not be illuminated by the second illumination device when the fundus image includes a lesion.

It is thereby possible to prevent going to the trouble of capturing a pupil image in order to determine whether or not there is a lesion, also in the case where it can be determined from a fundus image that there is a lesion in an animal. It is thereby possible to eliminate wasteful processing and the accumulation of information.

Furthermore, cover glass that covers the fundus imaging camera, between the fundus imaging camera and the animal, and a cover glass cleaning device that cleans the cover glass when the number of times the identification unit has not been able to identify the individual animal is equal to or greater than a predetermined number of times may be additionally provided.

Thus, in the case where the identification of an individual animal fails a predetermined number of times or more, because the cover glass is cleaned, it is possible to suppress the failure of individual identification after the cover glass has been cleaned.

Furthermore, a feeding system according to an aspect of the present disclosure is a feeding system that feeds an animal using a fundus image and a pupil image of the animal captured by a camera system, the camera system being provided with: a first illumination device that illuminates an eyeball of the animal; a fundus imaging camera that captures the fundus image of the eyeball illuminated by the first illumination device; a second illumination device that illuminates an eyeball of the animal at the same timing as the first illumination device; a pupil imaging camera that captures the pupil image of the eyeball illuminated by the second illumination device; an output circuit that outputs the fundus image as identification information of the animal, and outputs the pupil image as biological information of the animal corresponding to the identification information; an estimation unit that estimates the concentration of vitamin A in blood of the animal using the pupil image; and an interface that outputs a signal for switching the composition of feed, corresponding to the concentration of the vitamin A estimated by the estimation unit.

Thus, the vitamin A blood concentration of an animal can be acquired while that individual animal is appropriately identified, and feed to be given to that animal can be made to have the optimum feed composition ratio corresponding to the vitamin A blood concentration of that animal. For example, a cow can be fed with the optimum feed composition ratio for improving the meat quality without a severe illness such as blindness occurring.

An imaging device according to an aspect of the present disclosure includes: a first camera that captures a first image of a first eye illuminated by infrared light radiated from an infrared light radiator, an animal having the first eye and a second eye that is different from the first eye; a second camera, the distance between an objective lens of the first camera and the first eye being less than the distance between an objective lens of the second camera and the second eye; a decider that decides which one of processes including a first process and a second process is to be executed, each of the processes, when executed, being executed after the first image is captured; and an outputter that outputs a plurality of images in the second process, in the first process, the first camera capturing an additional first image of the first eye illuminated by additional infrared light radiated from the infrared light radiator, in the second process, (i) the first camera capturing a second image of the first eye illuminated by first white light radiated from a first white light radiator, (ii) the second camera capturing a third image of the second eye illuminated by second white light radiated from a second white light radiator, and (iii) the second camera capturing a fourth image of the second eye illuminated by the second white light, the plurality of images including the second image, the third image, and the fourth image, and the time interval between the first image being captured and the additional first image being captured being greater than the time interval between the third image being captured and the fourth image being captured.

A decider that decides the one process, based on luminance data of a pixel of the first image, may be additionally included.

Hereinafter, embodiments will be described in a specific manner with reference to the drawings.

It should be noted that the embodiments described hereinafter all represent general or specific examples. The numerical values, the shapes, the materials, the constituent elements, the arrangement positions and modes of connection of the constituent elements, the steps, and the order of the steps and the like given in the following embodiments are examples and are not intended to limit the present disclosure. Furthermore, from among the constituent elements in the following embodiments, constituent elements that are not mentioned in the independent claims indicating the most significant concepts are described as optional constituent elements. It should be noted that a cow means a domestic bovine animal, regardless of sex or age, in this disclosure.

### Embodiment 1

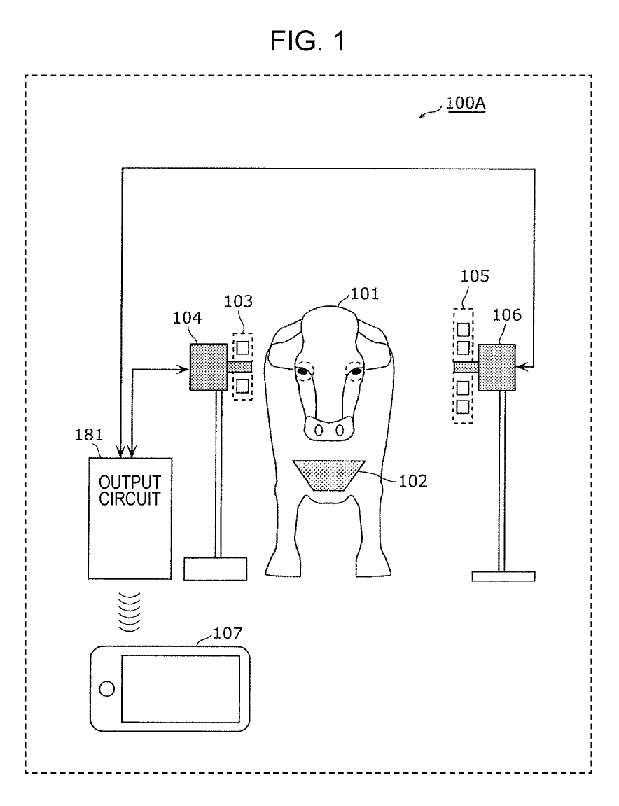

FIG. 1 depicts a camera system 100A in embodiment 1. The camera system 100A is a camera system that captures images of the eyeballs of an animal, and is provided with a first illumination device 103, a fundus imaging camera 104, a second illumination device 105, a pupil imaging camera 106, and an output circuit 181. The camera system 100A captures a fundus image and a pupil image of the animal, and outputs the captured fundus image and pupil image to a mobile terminal 107. FIG. 1 depicts a cow 101 as an example of the animal. Another example of the animal is a dog, a cat, or the like. That is, the camera system in the present disclosure captures a fundus image and a pupil image of the cow 101 as an example of the animal; however, that animal is not restricted to the cow 101 and may be another animal such as a dog or a cat. Hereinafter, the cow 101 will be described as an example of the animal.

The camera system 100A, for example, is installed adjacent to a water drinking station in a cow pen in which ordinarily four or five cows are reared in a cattle barn of a farmer. Furthermore, the camera system 100A captures images of both eyeballs while the cow 101 is drinking water from inside a water cup 102, or at a timing at which the water drinking has been completed, at night when there is mainly no external light.

### (First Illumination Device 103 and Second Illumination Device 105)

The first illumination device 103 illuminates an eyeball of the animal. The second illumination device 105 illuminates an eyeball of the animal at the same timing as the first illumination device 103. The same timing in the present specification means that the illumination timing of the first illumination device 103 and the illumination timing of the second illumination device 105 are within 0.3 sec. That is, the second illumination device 105 emits light within 0.3 sec from the point in time at which the first illumination device 103 emitted light. It should be noted that the point in time at which the first illumination device 103 emitted light is the point in time at which the first illumination device 103 started to emit light.

An example of the first illumination device 103 and the second illumination device 105 is at least one of a white illumination device and an infrared illumination device. That is, the first illumination device 103 in the present embodiment is an infrared illumination device or a white illumination device, and the second illumination device 105 is a white illumination device. It should be noted that the white illumination device emits white light when turned on, and the infrared illumination device emits infrared light when turned on. The first illumination device 103 may be incorporated in the fundus imaging camera 104 as a single unit. Furthermore, the second illumination device 105 may be incorporated in the pupil imaging camera 106 as a single unit.

The first illumination device 103 may have an optical axis similar to that of the fundus imaging camera 104. Furthermore, the second illumination device 105 may have an optical axis similar to that of the pupil imaging camera 106.

### (Fundus Imaging Camera 104)

The fundus imaging camera 104 captures a fundus image of the eyeball of the animal illuminated by the first illumination device 103. An example of the fundus imaging camera 104 is a color camera in the case where the first illumination device 103 is a white illumination device. An example of the fundus imaging camera 104 is an infrared camera in the case where the first illumination device 103 is an infrared illumination device. Furthermore, the fundus imaging camera 104 may have a function as a color camera and a function as an infrared camera, and these functions may be switched. The fundus imaging camera 104 functions as a color camera and functions as an infrared camera by switching filters that restrict the wavelength of light that is incident upon an image sensor, for example. The fundus imaging camera 104 functions as a color camera in the case where white light is radiated from the first illumination device 103, and functions as an infrared camera in the case where infrared light is radiated from the first illumination device 103.

### (Pupil Imaging Camera 106)

The pupil imaging camera 106 captures a pupil image of the eyeball of the animal illuminated by the second illumination device 105. An example of the pupil imaging camera 106 is a color camera in the case where the second illumination device 105 is a white illumination device. It should be noted that, similar to the fundus imaging camera 104, the pupil imaging camera 106 may have a function as a color camera and a function as an infrared camera, and these functions may be switched. When the sensitivity band for the image sensor is set so as to include visible light to infrared light, for example, and the subject is illuminated in a darkroom state such as at night, the pupil imaging camera 106 functions as a color camera and functions as an infrared camera by switching filters that restrict the wavelength of illumination light. The pupil imaging camera 106 functions as a color camera in the case where white light is radiated from the second illumination device 105, and functions as an infrared camera in the case where infrared light is radiated from the second illumination device 105.

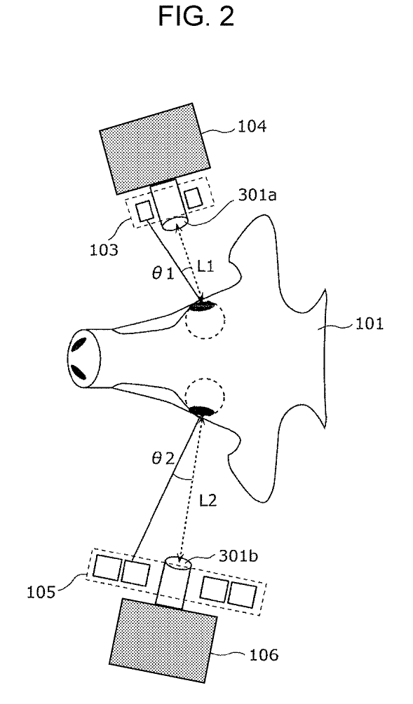

FIG. 2 depicts an example of the arrangement positions of the fundus imaging camera 104 and the pupil imaging camera 106 in embodiment 1. In FIG. 2, the fundus imaging camera 104 is arranged facing the right eyeball, and the pupil imaging camera 106 is arranged facing the left eyeball.

It is necessary for the fundus imaging camera 104 to observe the retina from the pupil in a state in which the light that is output from the first illumination device 103 has reached the retina behind the pupil. Consequently, an angle θ1 formed by the illumination optical axis of the first illumination device 103 and the imaging optical axis of the fundus imaging camera 104 may be small. The illumination optical axis of the first illumination device 103 and the imaging optical axis of the fundus imaging camera 104 may be more or less the same, for example, 0°≦θ1≦15°.

The color of the surface of the cornea of the eyeball included in the pupil image and the constriction of the pupil (pupil constriction) due to a pupillary (light) reflex are equivalent to the biological information of the animal. Consequently, it is sufficient as long as the pupil imaging camera 106 is able to capture an image of the surface of the eyeball. Therefore, the second illumination device 105 does not have to be able to illuminate to the rear of the eyeball, and therefore an angle θ2 formed by the illumination optical axis of the second illumination device 105 and the imaging optical axis of the pupil imaging camera 106 does not have to be as small. Consequently, in the present embodiment, it is necessary for the condition θ1≦θ2 to be satisfied.

Furthermore, the fundus imaging camera 104 has a first objective lens 301a and the pupil imaging camera 106 has a second objective lens 301b. Here, in the case where the first objective lens 301a of the fundus imaging camera 104 and the second objective lens 301b of the pupil imaging camera 106 are implemented as the same optical system, the positional relationship between the fundus imaging camera 104 and the pupil imaging camera 106 satisfies the following condition. That is, when the distance between the first objective lens 301a of the fundus imaging camera 104 and the surface of an eyeball is L1, and the distance between the second objective lens 301b of the pupil imaging camera 106 and the surface of an eyeball is L2, the condition L1<L2 is satisfied.

This is because the position of the fundus of the animal is located away from the surface of the pupil by approximately 5 cm to 10 cm. In order for images of the fundus and the pupil to be captured at approximately the same viewing angle, it is necessary for the fundus imaging camera 104 to be positioned closer to animal than the pupil imaging camera 106. Furthermore, due to a lens effect caused by the lens of the eye, the fundus image is present extending to an almost infinitely distant position. The cause lies in that, when observed, the fundus image is viewed through the pupil as a window and therefore the observation range becomes extremely narrow. In order to view the fundus image with a wide range, the apparent diameter of the pupil that constitutes a window should be as large as possible. Therefore, it is necessary for the fundus imaging camera 104 to be positioned closer to animal than the pupil imaging camera 106.

### (Output Circuit 181)

The output circuit 181 outputs the fundus image as identification information of the animal, and outputs the pupil image as biological information of the animal corresponding to the identification information. The output circuit 181 in the present embodiment outputs the fundus image and the pupil image to the mobile terminal 107, but, for example, may output that fundus image and pupil image to a display, a control circuit, or the like. It should be noted that the mobile terminal 107 is a tablet terminal, a smartphone, a personal computer, or the like of a user such as a fattening farmer.

The user is able to acquire the biological information of the animal while appropriately identifying that individual animal, by using the fundus image and the pupil image that have been output to the mobile terminal 107.

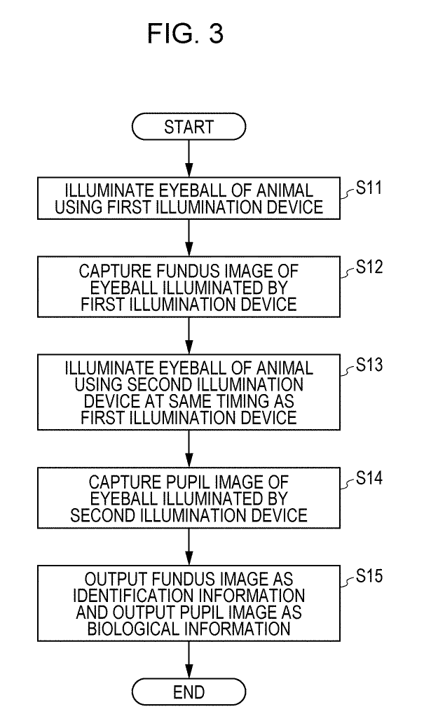

FIG. 3 is a flowchart depicting a processing operation of the camera system 100A in the present embodiment, namely an imaging method for capturing images of the eyeballs of the animal.

### (Step S11)

First, the first illumination device 103 illuminates an eyeball of the animal.

### (Step S12)

The fundus imaging camera 104 captures a fundus image of the eyeball illuminated by the first illumination device 103.

### (Step S13)

The second illumination device 105 illuminates an eyeball of that animal at the same timing as the first illumination device 103.

### (Step S14)

The pupil imaging camera 106 captures a pupil image of the eyeball illuminated by the second illumination device 105.

### (Step S15)

The output circuit 181 outputs that fundus image as identification information of the animal, and outputs that pupil image as biological information of the animal corresponding to that identification information.

### Effect of Embodiment 1

The camera system 100A in the present embodiment is a camera system that captures images of the eyeballs of the animal, and is provided with the first illumination device 103, the fundus imaging camera 104, the second illumination device 105, the pupil imaging camera 106, and the output circuit 181. The first illumination device 103 illuminates an eyeball of the animal. The fundus imaging camera 104 captures a fundus image of the eyeball illuminated by the first illumination device 103. The second illumination device 105 illuminates an eyeball of the animal at the same timing as the first illumination device 103. The pupil imaging camera 106 captures a pupil image of the eyeball illuminated by the second illumination device 105. The output circuit 181 outputs the fundus image as identification information of the animal, and outputs the pupil image as biological information of the animal corresponding to that identification information.

Thus, by using two cameras, a fundus image constituting identification information of the animal and a pupil image constituting biological information of that animal can be acquired at the same time. As a result, the identification of the animal and the acquisition of biological information can be carried out quickly. Furthermore, in the camera system 100A, the second illumination device 105 illuminates an eyeball of the animal at the same timing as the first illumination device 103. Consequently, the pupil image of the eyeball illuminated by the second illumination device 105 can be appropriately captured even if pupil constriction is about to start or even if the animal is about to run away due to the eyeball being illuminated by the first illumination device 103 in order to capture the fundus image, for example. Consequently, in the camera system 100A in the present embodiment, the biological information of the animal can be acquired while that individual animal is appropriately identified.

Furthermore, in the present embodiment, the first illumination device 103 is an infrared illumination device or a white illumination device, and the second illumination device 105 is a white illumination device.

Thus, an infrared image or a color image in which a clear blood vessel pattern is depicted to a degree enabling the animal to be identified can be acquired as the fundus image, and a color image enabling the pupil color to be specified can be acquired as the pupil image. That is, the individual identification of the animal and the acquisition of biological information can be carried out appropriately.

Furthermore, in the present embodiment, the second illumination device 105 emits light within 0.3 sec from the point in time at which the first illumination device 103 emitted light.

Thus, the biological information of the animal can be acquired while that individual animal is appropriately identified, with reduced effect from pupil constriction or the animal running away due to the eyeballs being illuminated.

Furthermore, in the present embodiment, when the angle formed by the illumination optical axis of the first illumination device 103 and the imaging optical axis of the fundus imaging camera 104 is θ1, and the angle formed by the illumination optical axis of the second illumination device 105 and the imaging optical axis of the pupil imaging camera 106 is θ2, the condition θ1≦θ2 is satisfied.

Thus, the fundus imaging camera 104 is able to observe the retina from the pupil in a state in which the light that is output from the first illumination device 103 has reached the retina behind the pupil. As a result, the blood vessel pattern on the retina illuminated by the first illumination device 103 can be appropriately captured as a clear fundus image.

Furthermore, in the present embodiment, the fundus imaging camera 104 has the first objective lens 301a, and the pupil imaging camera 106 has the second objective lens 301b. Also, when the distance between the first objective lens 301a and the position of the surface of an eyeball of the animal is L1, and the distance between the second objective lens 301b and the position of the surface of an eyeball of the animal is L2, the condition L1<L2 is satisfied.

The position of the fundus of the animal is located further to the rear than the pupil surface, and therefore, because L1<L2, images of the fundus and the pupil can be captured at approximately the same viewing angle.

It should be noted that, in order to satisfy L1<L2, “the distance between the water cup 102 and the objective lens of the fundus imaging camera 104”<“the distance between the water cup 102 and the objective lens of the pupil imaging camera 106” may be implemented, as depicted in FIG. 1. The water cup 102 may be a container that contains food.

### Embodiment 2

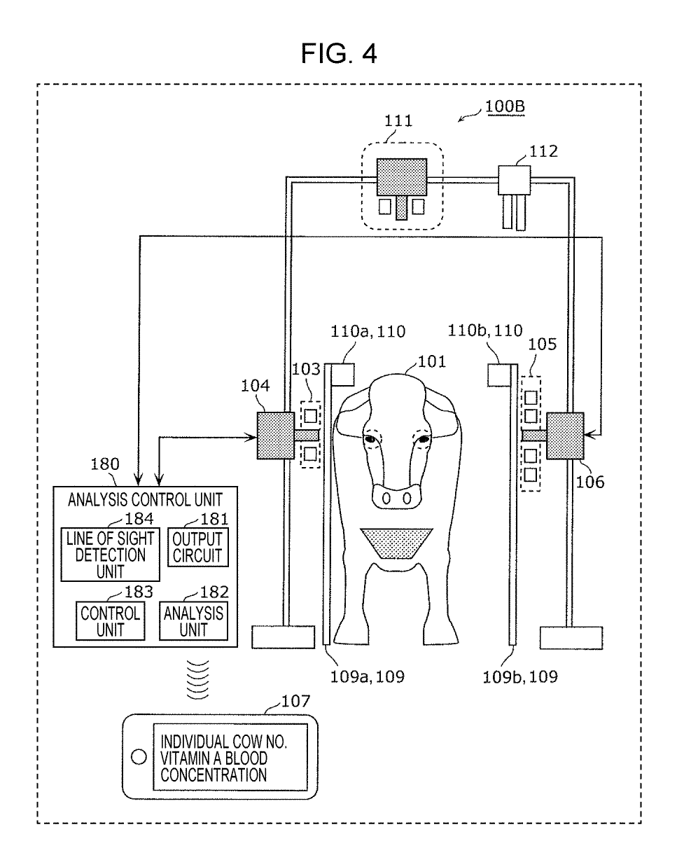

FIG. 4 depicts a camera system 100B in embodiment 2. The camera system 100B is provided with the constituent elements included in the camera system 100A of embodiment 1. In addition, the camera system 100B is provided with cover glass 109, a cover glass cleaning device 110, an individual authentication camera 111, an antenna 112 of a radio frequency identifier (RFID), an analysis unit 182, a control unit 183, and a line of sight detection unit 184. It should be noted that, in the present embodiment, an analysis control unit 180 is configured of that analysis unit 182, control unit 183, and line of sight detection unit 184, and the output circuit 181 of embodiment 1. Furthermore, the camera system 100B in the present embodiment, similar to embodiment 1, captures a fundus image and a pupil image of the cow 101 as an example of the animal; however, that animal is not restricted to the cow 101 and may be another animal such as a dog or a cat. Hereinafter, the cow 101 will be described as an example of the animal.

### (Cover Glass 109)

The cover glass 109 includes first cover glass 109a for the fundus imaging camera 104, and second cover glass 109b for the pupil imaging camera 106. The first cover glass 109a covers the fundus imaging camera 104, between the fundus imaging camera 104 and the cow 101. Similarly, the second cover glass 109b covers the pupil imaging camera 106, between the pupil imaging camera 106 and the cow 101. The first cover glass 109a and the second cover glass 109b may be a single sheet of cover glass.

### (Cover Glass Cleaning Device 110)

The cover glass cleaning device 110 includes a first cover glass cleaning device 110a and a second cover glass cleaning device 110b. The first cover glass cleaning device 110a has a wiper, for example, and cleans the first cover glass 109a. Similarly, the second cover glass cleaning device 110b has a wiper, for example, and cleans the second cover glass 109b. The first cover glass cleaning device 110a and the second cover glass cleaning device 110b may be a single cleaning device.

### (Individual Authentication Camera 111)

The individual authentication camera 111 is a preliminary means for carrying out individual authentication of the cow 101, and photographs the number of the ear tag of the cow.

### (Antenna 112)

The antenna 112, similar to the individual authentication camera 111, is a preliminary means for carrying out individual authentication of the cow 101, and is an antenna for reading a signal from an RFID tag attached to the cow 101.

### (Control Unit 183)

The control unit 183 controls the overall operation of the camera system 100B.

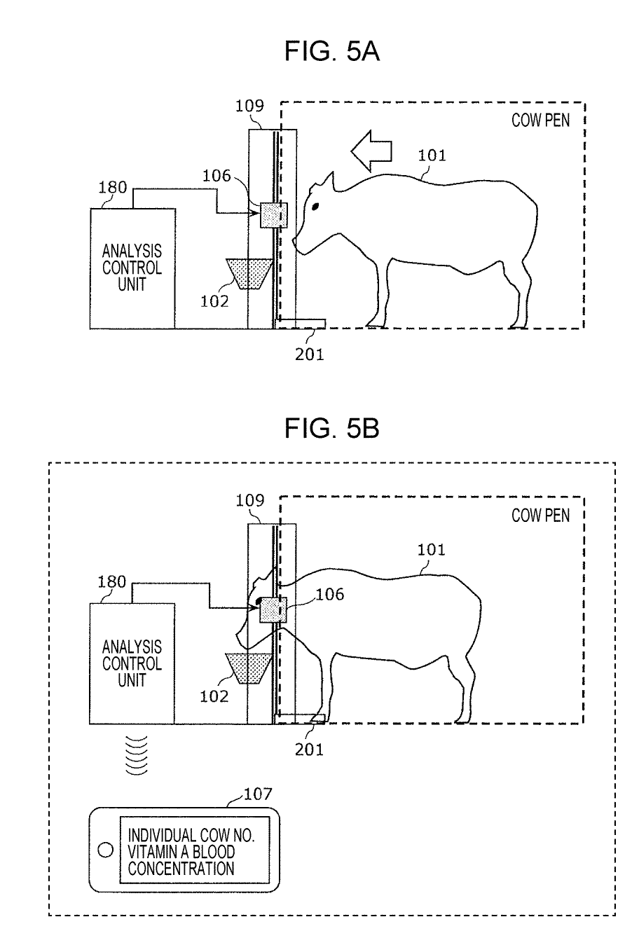

FIGS. 5A and 5B depict a usage example of the camera system 100B. FIG. 5A depicts a state in which the cow 101 is approaching the water drinking station from inside the cattle barn at night.

As depicted in FIG. 5B, the cow 101 is drinking water from the water cup 102. This state is detected by a pressure sensor 201. When this state is detected, the operation of the camera system 100B starts in order to capture images of the eyeballs of the cow. That is, the control unit 183 starts control of the first illumination device 103, the second illumination device 105, the fundus imaging camera 104, and the pupil imaging camera 106 in accordance with a signal from the pressure sensor 201. Specifically, during this water intake period for the cow 101, the first illumination device 103 and the second illumination device 105 turn on and operate in accordance with an instruction from the control unit 183. In addition, during this water intake period, the fundus imaging camera 104 and the pupil imaging camera 106 capture color images of the left and right eyeballs of the cow 101 in accordance with an instruction from the control unit 183.

The control unit 183 causes the analysis unit 182 to carry out and record an analysis that accompanies the image processing of the acquired images. Information indicating the result of that analysis is, as appropriate, notified to the mobile terminal 107 such as a smartphone or a tablet terminal, and is displayed on the display of that mobile terminal 107.

In this way, in the camera system 100B, the health condition of the cow 101 is recorded with the acquisition of the pupil image, which is conventionally carried out with an imaging device being pressed up against an eyeball of the cow 101 by a livestock raiser or a veterinarian, being realized completely automatically at night in a non-contact manner without the cow 101 being touched at all. The individual identification of the cow 101 may also be carried out at the same time by means of a technology such as image sensing or an RFID tag, and may be recorded together with the pupil image.

### (Specific Configurations of Illumination Devices and Cameras)

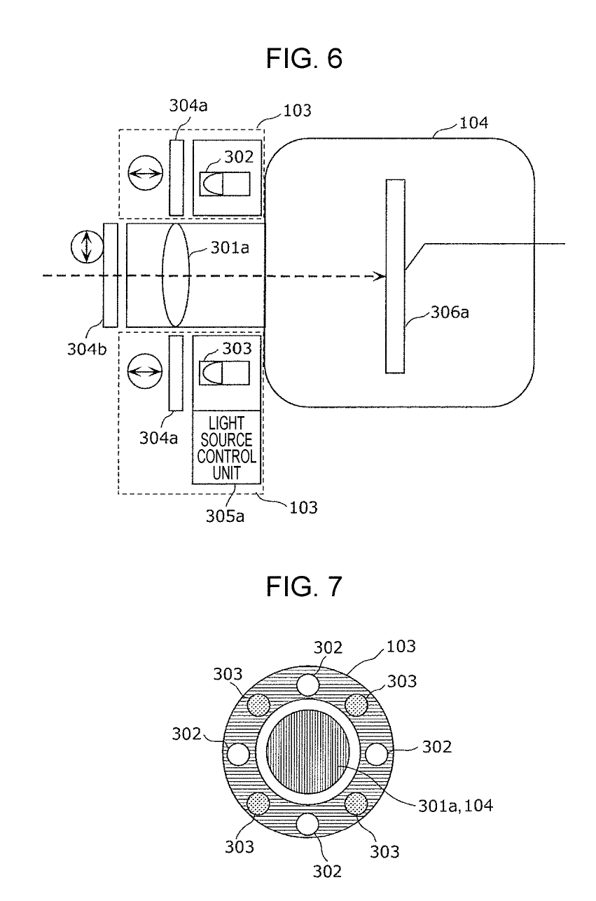

FIG. 6 depicts an example of a configuration of the first illumination device 103 and the fundus imaging camera 104. The fundus imaging camera 104 is provided with the first objective lens 301a and a first image sensor 306a. An example of the first image sensor 306a is a single-plate color image sensor.

An example of the first illumination device 103 is provided with a white illumination device made up of a plurality of white LEDs 302, an infrared illumination device made up of a plurality of infrared LEDs 303, and a light source control unit 305a.

FIG. 7 depicts an example of the first illumination device 103 and the fundus imaging camera 104 seen from the optical axis direction. The light source control unit 305a controls the turning on and off for the plurality of white LEDs 302 and the plurality of infrared LEDs 303, in accordance with an instruction from the control unit 183.

As depicted in FIG. 7, the plurality of white LEDs 302 and the plurality of infrared LEDs 303 are arranged in such a way as to surround the periphery of the first objective lens 301a. The illumination optical axes of the plurality of white LEDs 302 and the plurality of infrared LEDs 303 are substantially coaxial with the imaging optical axis of the fundus imaging camera 104. Here, substantially coaxial means that the angle formed between the illumination optical axis and the imaging optical axis is within approximately 15°.

Each white LED 302 and each infrared LED 303 may be provided with a first linear polarizing plate 304a. The first linear polarizing plate 304a is arranged on the front surface of each white LED 302 and each infrared LED 303. The fundus imaging camera 104 may be provided with a second linear polarizing plate 304b. The second linear polarizing plate 304b is arranged on the front surface of the fundus imaging camera 104 (specifically, the first objective lens 301a).

The first linear polarizing plate 304a has a polarization axis of 0° (horizontal). The second linear polarizing plate 304b has a polarization axis of 90° (vertical). Mirror surface reflection of the illumination from the cornea or the like of an eyeball can thereby be eliminated.

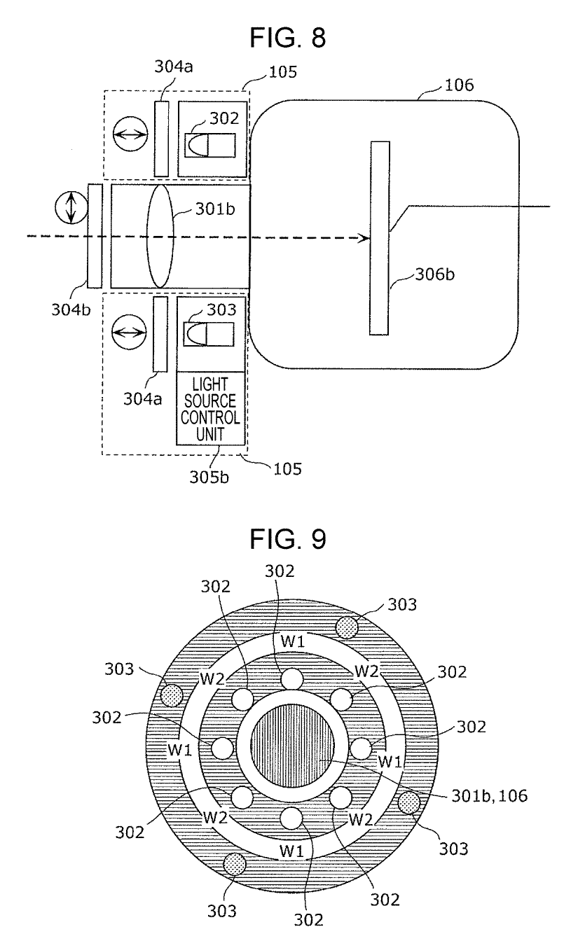

FIG. 8 depicts an example of a configuration of the second illumination device 105 and the pupil imaging camera 106. The pupil imaging camera 106 is provided with the second objective lens 301b and a second image sensor 306b. An example of the second image sensor 306b is a single-plate color image sensor.

An example of the second illumination device 105 is provided with a white illumination device made up of a plurality of the white LEDs 302, an infrared illumination device made up of a plurality of the infrared LEDs 303, and a light source control unit 305b.

FIG. 9 depicts an example of the second illumination device 105 and the pupil imaging camera 106 seen from the optical axis direction. The light source control unit 305b controls the turning on and off for the plurality of white LEDs 302 and the plurality of infrared LEDs 303, in accordance with an instruction from the control unit 183.

As depicted in FIG. 9, the plurality of white LEDs 302 and the plurality of infrared LEDs 303 are arranged in such a way as to surround the periphery of the second objective lens 301b.

In the second illumination device 105 also, similar to the first illumination device 103, each white LED 302 and each infrared LED 303 may be provided with the first linear polarizing plate 304a. The first linear polarizing plate 304a is arranged on the front surface of each white LED 302 and each infrared LED 303. The pupil imaging camera 106, similar to the fundus imaging camera 104, may be provided with the second linear polarizing plate 304b. The second linear polarizing plate 304b is arranged on the front surface of the pupil imaging camera 106 (specifically, the second objective lens 301b).

Furthermore, the second illumination device 105 is made up of two types of concentric circular ring illumination devices arranged in such a way as to surround the second objective lens 301b of the pupil imaging camera 106. A ring illumination device having a small radius is a white illumination device, and the plurality of white LEDs 302 are arranged in this white illumination device. Each of the plurality of white LEDs 302 belongs to a channel W1 or W2. A ring illumination device having a large radius is an infrared illumination device, and the plurality of infrared LEDs 303 are arranged in this infrared illumination device. The light source control unit 305b is able to turn the plurality of white LEDs 302 on and off for each channel, and is also able to turn the plurality of infrared LEDs 303 on and off, according to a signal from the control unit 183.

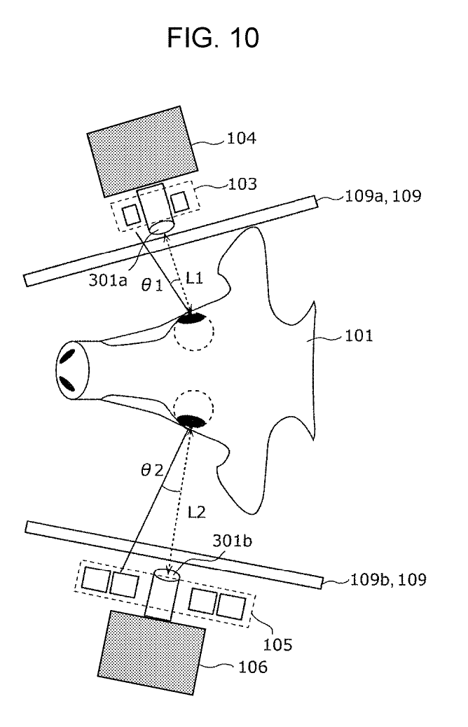

FIG. 10 depicts an example of the arrangement positions of the fundus imaging camera 104 and the pupil imaging camera 106 in embodiment 2. In FIG. 10, the fundus imaging camera 104 is arranged facing the right eyeball with the first cover glass 109a therebetween, and the pupil imaging camera 106 is arranged facing the left eyeball with the second cover glass 109b therebetween.

Furthermore, in the present embodiment also, similar to embodiment 1, an angle θ1 formed by the illumination optical axis of the first illumination device 103 and the imaging optical axis of the fundus imaging camera 104 may be small. The illumination optical axis of the first illumination device 103 and the imaging optical axis of the fundus imaging camera 104 may be more or less the same. Furthermore, it is not necessary for an angle θ2 formed by the illumination optical axis of the second illumination device 105 and the imaging optical axis of the pupil imaging camera 106 to be as small. Consequently, in the present embodiment also, similar to embodiment 1, it becomes necessary for the condition θ1≦θ2 to be satisfied.

Furthermore, in the present embodiment also, similar to embodiment 1, the positional relationship of the fundus imaging camera 104 and the pupil imaging camera 106 satisfies the condition L1<L2.

In the present embodiment, the first illumination device 103 is provided with a white illumination device and an infrared illumination device; however, the first illumination device 103 may not be provided with a white illumination device. In this case, the camera system 100B is additionally provided with an infrared illumination device.

### (Line of Sight Detection Unit 184)

The line of sight detection unit 184 detects the line of sight of the cow 101. The fundus imaging camera 104 captures a fundus image for detecting the line of sight of an eyeball illuminated by the infrared illumination device. The line of sight detection unit 184 detects the line of sight of the cow 101 using that fundus image for detecting the line of sight. The first illumination device 103 and the second illumination device 105 illuminate the eyeballs on the basis of the detected line of sight of the cow 101. The fundus imaging camera 104 captures a fundus image of those eyeballs, and the pupil imaging camera 106 captures a pupil image of those eyeballs. Furthermore, in the present embodiment, the first illumination device 103 and the second illumination device 105 illuminate the eyeballs when the detected line of sight of the cow 101 is the same as the imaging optical axis of the fundus imaging camera 104.

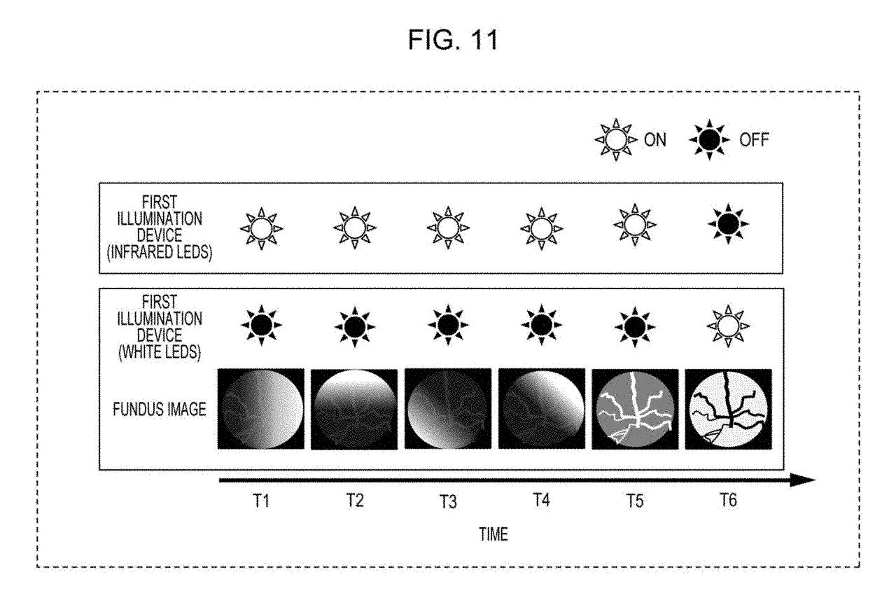

FIG. 11 depicts timings for the illumination performed by the first illumination device 103 and the imaging performed by the fundus imaging camera 104.

The plurality of infrared LEDs 303 (infrared illumination device) in the first illumination device 103 emit light in accordance with an instruction from the control unit 183, and illuminate an eyeball of the cow 101 with infrared light. At such time, the fundus imaging camera 104 continuously captures fundus images of the eyeball of the cow 101 illuminated by the infrared light. Each of the fundus images continuously captured at such time is an aforementioned fundus image for detecting the line of sight, and is an infrared image. Hereinafter, these fundus images are also referred to as infrared fundus images. The line of sight detection unit 184 continuously detects the line of sight of the eyeball of the cow 101 without being sensed by the cow 101, on the basis of these continuously captured fundus images (infrared fundus images). The line of sight detection unit 184 then detects a timing at which the fundus is directly facing the fundus imaging camera 104, in other words, a timing at which the line of sight of the eyeball is directed toward the fundus imaging camera 104. Immediately after this detected timing, the plurality of white LEDs 302 (white illumination device) in the first illumination device 103 illuminate the eyeball of the cow 101 with white light by emitting light in accordance with an instruction from the control unit 183. In addition, at such time, the fundus imaging camera 104 acquires a fundus image of the eyeball illuminated by the white light. The fundus image at such time is a color image, and, hereinafter, the fundus image at such time is also referred to as a color fundus image.

The fundus imaging camera 104 captures an image of the retina at the rear the eyeball, not the pupil. Consequently, the line of sight detection unit 184 is not able to detect the line of sight from the infrared fundus images in the usual sense. However, a satisfactory fundus image is not obtained unless a state in which the pupil is directly facing the fundus imaging camera 104 is detected and captured. Thus, in the present embodiment, the eyeball is tracked while infrared light is continuously radiated, and the acquisition of infrared fundus images and image processing are continuously carried out. Waiting is then performed until a timing at which an infrared fundus image is evenly bright with there being no dark regions and the retina blood vessel pattern can be clearly seen. At this timing, a state has been entered in which the pupil is directly facing the fundus imaging camera 104, in other words, a state in which the line of sight is directed toward the fundus imaging camera 104. That is, the line of sight detection unit 184 detects the line of sight of the cow 101 in accordance with the clarity of the infrared fundus images.

As depicted in FIG. 11, the infrared fundus images are unclear at time slots T1, T2, T3, and T4. However, a clear infrared fundus image is obtained at the point in time of time slot T5. This clear infrared fundus image is an image in which the infrared fundus image is evenly bright with there being no dark regions and the retina blood vessel pattern can be clearly seen. When this clear infrared fundus image is obtained, the line of sight detection unit 184 detects that the line of sight of the eyeball is directed toward the fundus imaging camera 104. The control unit 183 causes the first illumination device 103 to switch the emitted light from infrared light to white light at time slot T6 immediately after the timing at which the aforementioned detection was carried out. In addition, the control unit 183 causes the fundus imaging camera 104 to capture a fundus image of the eyeball being illuminated with white light, as a color fundus image at that time slot T6.

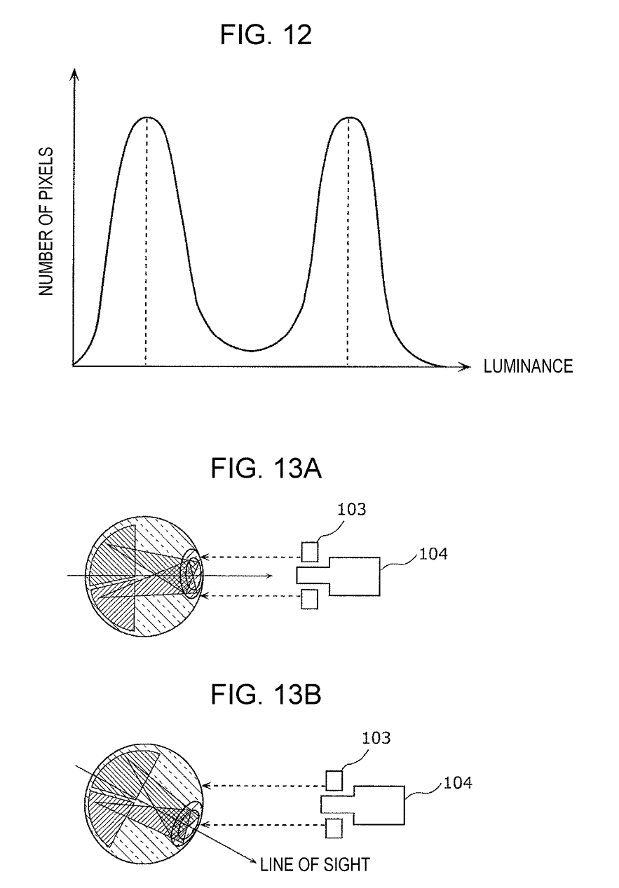

FIG. 12 depicts an example of a histogram of a clear infrared fundus image.

A histogram of the luminance of each pixel in a clear infrared fundus image has two peaks as depicted in FIG. 12. It should be noted that, in the histogram of FIG. 12, the horizontal axis indicates luminance and the vertical axis indicates the number of pixels. Conversely, in the case where the histogram only has one peak, or in the case where there are three or more peaks, the infrared fundus image corresponding to that histogram is unclear. The line of sight detection unit 184 performs image processing on an infrared fundus image, and thereby determines whether or not the histogram corresponding to that infrared fundus image has two peaks. In the case where there are two peaks, that is, in the case where an infrared fundus image is clear, when that infrared fundus image is captured, the line of sight detection unit 184 detects that the line of sight of the eyeball of the cow 101 is directed toward the fundus imaging camera 104.

FIGS. 13A and 13B depict a relationship between the line of sight of the eyeball and the first illumination device 103 and fundus imaging camera 104.

As depicted in FIG. 13B, in the case where the line of sight is not directed toward the fundus imaging camera 104, it is difficult for the light from the first illumination device 103 to reach the fundus via the pupil. In addition, even if that light reaches the fundus, it is difficult for the light reflected from that fundus to reach the fundus imaging camera 104.

However, as depicted in FIG. 13A, when the line of sight is directed toward the fundus imaging camera 104, that is, when the pupil is directly facing the fundus imaging camera 104, the light from the first illumination device 103 easily reaches the fundus via the pupil. In addition, the light reflected from that fundus can easily reach the fundus imaging camera 104. As a result, a clear infrared fundus image can be obtained when the line of sight is directed toward the fundus imaging camera 104. It should be noted that, at such time, a clear color fundus image can be obtained when white light is radiated onto the eyeball.

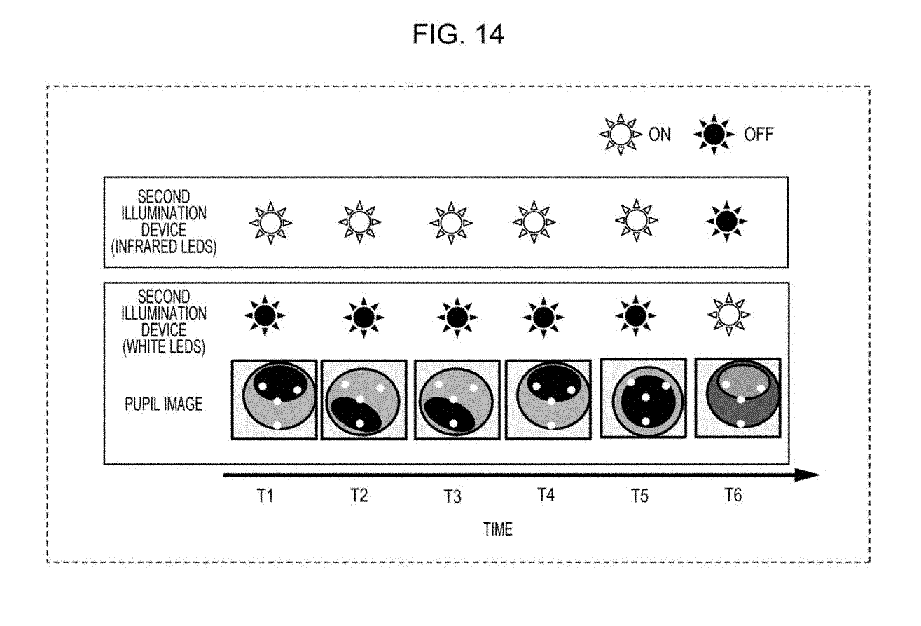

FIG. 14 depicts timings for the illumination performed by the second illumination device 105 and the imaging performed by the pupil imaging camera 106. Specifically, FIG. 14 depicts timings for the illumination performed by the second illumination device 105 and the imaging performed by the pupil imaging camera 106 with respect to the other eyeball in the same time slots T1 to T6 as in FIG. 11. It should be noted that the second illumination device 105 illuminates the eyeball with infrared light, and switches the light used for illumination from infrared light to white light. When the eyeball is being illuminated with that white light, the pupil imaging camera 106 captures a pupil image of that eyeball. The pupil image captured at such time is a color image.

The line of sight of the eyeball is directed in numerous directions in time slots T1 to T6, and the line of sight has deviated from the pupil imaging camera 106 at the timings of time slots T1 to T4 and T6. The pupil of the eyeball is directly facing the pupil imaging camera 106 at the timing of time slot T5.

However, the capturing of a pupil image by the pupil imaging camera 106 in a state in which infrared light is off and white light is on is carried out at the timing of time slot T6 not T5, that is, at the same timing as the capturing of the fundus image (specifically, the color fundus image). This is due to the following reasons. Firstly, in the eyeball from which the fundus image is acquired, pupil constriction occurs due to the illumination of white light; however, the nervous system that governs pupil constriction sometimes also affects the pupil constriction of the other eyeball, and sometimes both eyeballs start pupil constriction at the same time. Secondly, it is necessary for imaging to be carried out with respect to the cow 101 before that cow 101 is startled by the illumination of white light onto the other eyeball and runs away from the water drinking station. In addition, the pupil image does not require as precise matching of the line of sight as the fundus image, and it is possible for the pupil color to be determined and the pupil constriction velocity to be determined even with a slightly slanted line of sight.

In this way, in the present embodiment, the first illumination device 103 and the second illumination device 105 illuminate the eyeballs with white light when the detected line of sight of the cow 101 is the same as the imaging optical axis of the fundus imaging camera 104. Furthermore, in the present embodiment, although the fundus image and the pupil image are captured at the same timing, the fundus image is preferentially captured. That is, a fundus image for carrying out individual authentication of the cow 101 with a first eyeball is first preferentially acquired, and thereafter a pupil image of the second eyeball is acquired. Furthermore, the same timing may be that the difference between the timing at which the fundus image is captured and the timing at which the pupil image is captured is 0 sec or greater and approximately 0.3 sec or less. This is because the time delay from the illumination of white light to the start of pupil constriction is of this extent in the case of the cow 101.

### (Analysis Unit 182)

The analysis unit 182 acquires the fundus image and the pupil image that are output from the output circuit 181, analyzes those images, and thereby estimates specified biological information such as the vitamin A blood concentration.

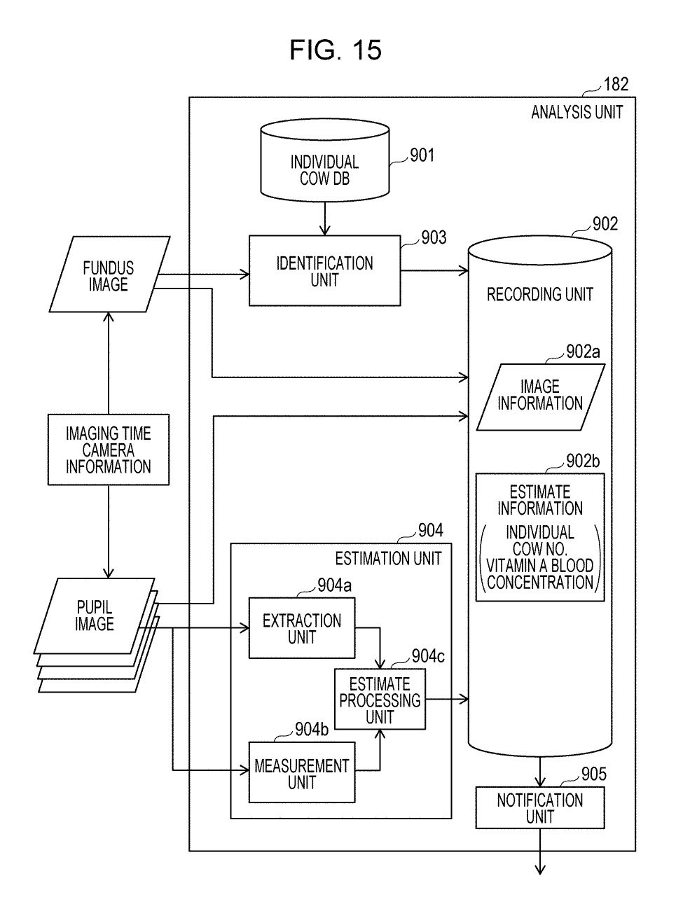

FIG. 15 is a block diagram of the analysis unit 182.

When acquiring the fundus image and the pupil image from the output circuit 181, the analysis unit 182 may acquire the fundus image and the pupil image having imaging times and camera information attached thereto, from the output circuit 181. An imaging time is the time at which imaging was performed by the fundus imaging camera 104, or the time at which imaging was performed by the pupil imaging camera 106. Furthermore, camera information is information for identifying the fundus imaging camera 104 or the pupil imaging camera 106.

This kind of analysis unit 182 is provided with an individual cow DB 901, a recording unit 902, an identification unit 903, an estimation unit 904, and a notification unit 905.

### (Individual Cow DB 901)

The individual cow DB 901 retains identification data in which the blood vessel patterns on the retinas of the eyeballs of each cow and the individual numbers of each cow (also referred to as the individual cow No.) are indicated in association with each other.

### (Identification Unit 903)

The identification unit 903 acquires a fundus image and identifies the individual cow 101 using that fundus image. It should be noted that identifying an individual animal such as the cow 101 is referred to as individual authentication or individual identification. Specifically, the identification unit 903 extracts the blood vessel pattern on the retina of an eyeball of the cow 101 from that fundus image. The identification unit 903 refers to the identification data retained in the individual cow DB 901, and thereby retrieves the individual number of the cow 101 associated with that extracted blood vessel pattern. When that individual number is found by retrieval, the identification unit 903 includes that individual number in estimate information 902b and stores such in the recording unit 902.

### (Recording Unit 902)

The recording unit 902 is a recording medium for retaining image information 902a and the estimate information 902b. The fundus image and the pupil image that are output from the output circuit 181 are indicated in association with each other in the image information 902a. It should be noted that the fundus image and the pupil image associated with each other in the image information 902a are images that have been obtained based on the same cow 101. The fundus image and the pupil image being images that have been obtained based on the same cow 101 is confirmed by means of the imaging times and camera information added to those images. That is, the imaging times added to those images indicate the same timings. In addition, the camera information added to those images indicates the fundus imaging camera 104 and the pupil imaging camera 106 which form a pair with each other.

### (Estimation Unit 904)

The estimation unit 904 estimates the concentration of vitamin A in the blood of the cow 101 using the pupil image. That is, the estimation unit 904 acquires the pupil image that is output from the output circuit 181, and estimates, as biological information, the vitamin A blood concentration of the cow 101 on the basis of that pupil image. This kind of estimation unit 904 is provided with an extraction unit 904a, a measurement unit 904b, and an estimate processing unit 904c.

### (Extraction Unit 904a)

The extraction unit 904a carries out color image processing on the pupil image. For example, the extraction unit 904a analyzes the ratio of RGB components of the pupil image, which is a color image. The extraction unit 904a thereby extracts color information indicating a pupil color from the pupil image.

### (Measurement Unit 904b)

The measurement unit 904b measures the pupil constriction velocity of the cow 101. Specifically, the second illumination device 105 once again illuminates an eyeball of the cow 101 within 0.3 sec from the point in time of having emitted light at the same timing as the first illumination device 103. The pupil imaging camera 106 captures a plurality of pupil images in accordance with the illumination performed by the second illumination device 105. For example, the pupil imaging camera 106 captures a plurality of pupil images by capturing images of the process in which the pupil constricts, at a frame rate of approximately 1/30 sec. The measurement unit 904b measures the pupil constriction velocity of the cow 101 using the plurality of pupil images. For example, the measurement unit 904b measures the pupil constriction velocity by dividing the amount of change in the area of the pupil from the start of pupil constriction to the end thereof, by the time from the start of that pupil constriction to the end thereof.

FIG. 11 depicts that the fundus imaging camera 104 continuously captures a plurality of fundus images of the eyeball of the cow 101 illuminated by infrared light. The interval during which the fundus imaging camera 104 captures fundus images (for example, the time intervals of T1 and T2 in FIG. 11, or the like) may be longer than the interval during which the pupil imaging camera 106 captures pupil images ( 1/30 sec in the aforementioned example).

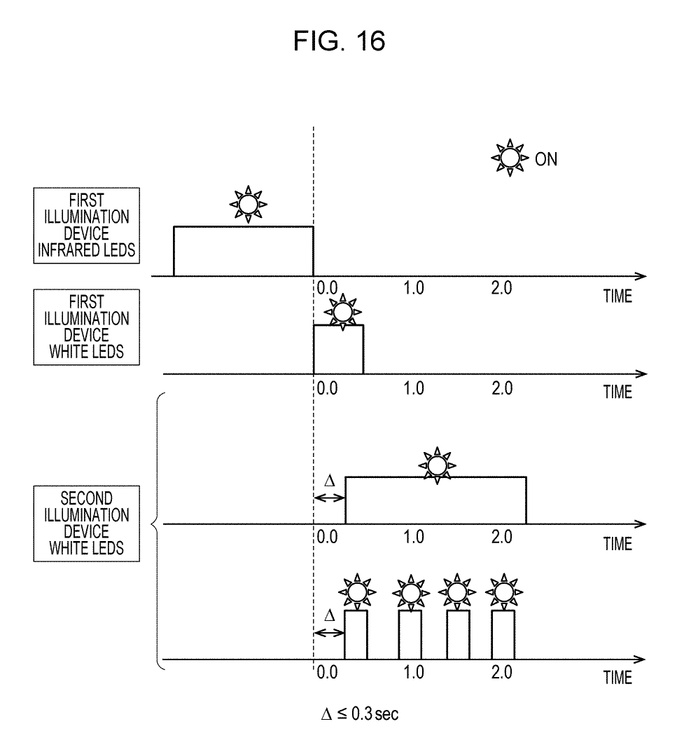

FIG. 16 is a drawing for describing a method for measuring the velocity of the constriction of a pupil (pupil constriction) due to light. According to prior research, the light reflex of an eyeball slows and the pupil constriction velocity slows when the vitamin A blood concentration is insufficient. Thus, by observing the pupil color using the second illumination device 105 and the pupil imaging camera 106 and observing the pupil constriction velocity at the same time, the vitamin A blood concentration can be estimated with greater accuracy. In the case of a human, pupil constriction appears in both eyeballs due to stimulation of an eyeball on one side; however, it is said that this does not always happen in the case of cows. Here, the light stimulation of an eyeball on one side is already being carried out by the first illumination device 103 in order to capture fundus images, and therefore the case where this light stimulation causes pupil constriction of the other eyeball will be described.

As depicted in FIG. 16, with respect to one eyeball of the cow 101, each infrared LED 303 of the first illumination device 103 turns on and then turns off, and the time at which each white LED 302 of the first illumination device 103 turns on is T=0 (sec). Pupil constriction of the other eyeball starts from this time T=0, and therefore each white LED 302 of the second illumination device 105 turns on within a fixed time Δ from time=0. It should be noted that the time Δ is equal to or less than 0.3 sec. When each white LED 302 of the second illumination device 105 is lit, the pupil imaging camera 106 captures the constriction of the pupil at each 1/30 (sec) continuously by means of video. A plurality of pupil images are thereby obtained as video. The measurement unit 904b performs image processing on this video, and thereby obtains the time from the constriction of the pupil starting to completing and calculates the pupil constriction velocity. Furthermore, each white LED 302 of the second illumination device 105 may repeatedly flash without being continuously lit, and the pupil imaging camera 106 may capture pupil images when each white LED 302 is lit. For example, as depicted in FIG. 9, each white LED 302 of the second illumination device 105 has a two-channel configuration made up of the channels W1 and W2. Consequently, each white LED 302 belonging to the channel W1 and each white LED 302 belonging to the channel W2 in the second illumination device 105 may emit light in an alternating manner. This light emission method has the benefit of it being possible to acquire the pupil color over a wider area in the case where the two types of measurement for pupil color and pupil constriction velocity are carried out at the same time in parallel.

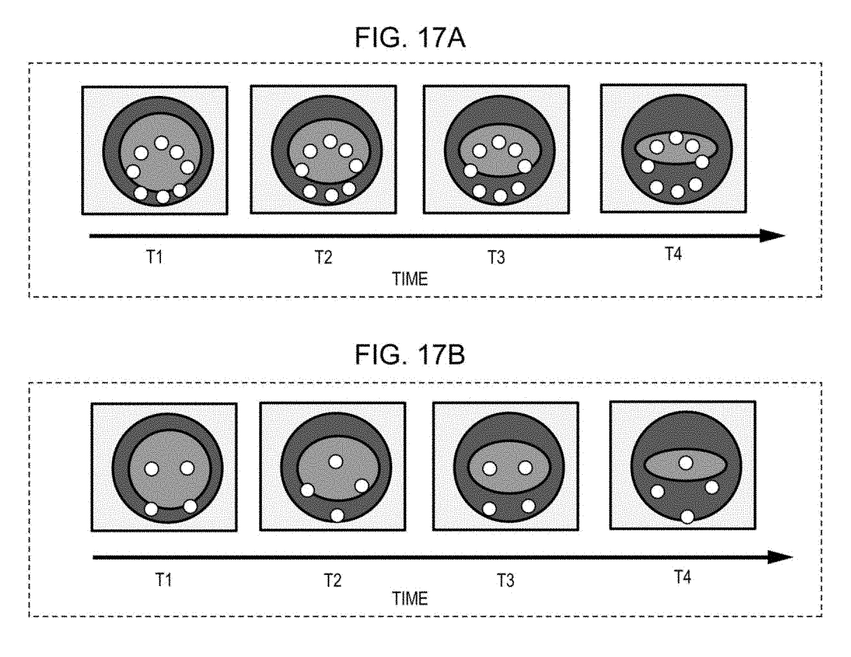

FIGS. 17A and 17B are explanatory diagrams of a light emission method for the second illumination device 105. As depicted in FIGS. 17A and 17B, pupil images are captured in each time slot from time slots T1 to T4. When all of the white LEDs 302 of the second illumination device 105 turn on in each time slot, as depicted in FIG. 17A, eight artifacts that are bright spots from mirror surface reflection of white light are also picked up on the surface of the cornea in the pupil images captured in each time slot. These artifacts normally cannot be completely eliminated even if the first linear polarizing plate 304a and the second linear polarizing plate 304b are used. These artifacts that are bright spots then become obstructive noise even in the case where the color of the pupil is averaged and even in the case where the area of the pupil is calculated in order to observe pupil constriction. However, as depicted in FIG. 17B, when each white LED 302 belonging to the channel W1 and each white LED 302 belonging to the channel W2 in the second illumination device 105 turn on in an alternating manner, those bright spots can be eliminated. That is, in two pupil images captured in adjacent time slots such as time slots T1 and T2, the positions of the bright spots are different. Thus, for example, from among pixels having the same coordinates included in each of the two pupil images, the pixels having a low luminance are used as the pixels for those coordinates in an image. By combining two pupil images according to this kind of method, it becomes possible for bright spots from mirror surface reflection of white light on the cornea to be eliminated and pupil color to be observed in all pupil images.

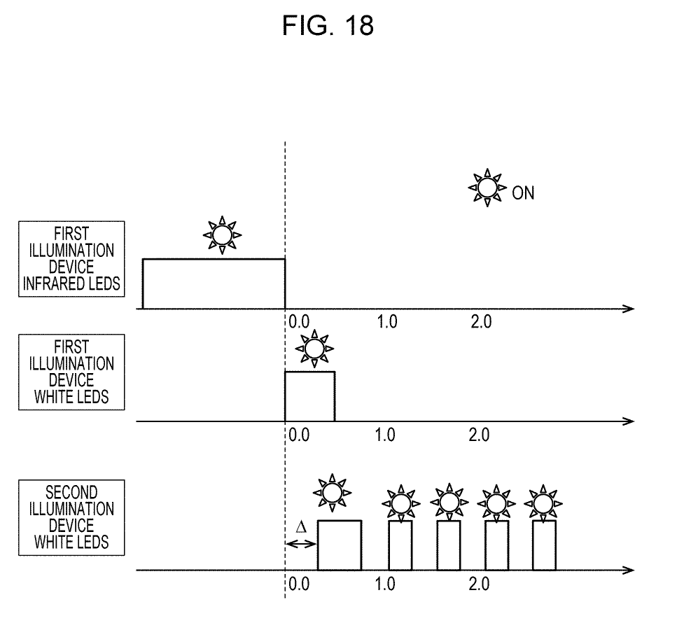

FIG. 18 is a drawing for describing another method for measuring the velocity of pupil constriction due to light.

FIG. 18 is used to describe the case where, even if the light stimulation of an eyeball on one side is already being carried out by the first illumination device 103 in order to capture a fundus image, pupil constriction in the other eyeball is induced by light stimulation that is independent from the aforementioned light stimulation of the eyeball on that one side.

As in FIG. 16, with respect to one eyeball of the cow 101, each infrared LED 303 of the first illumination device 103 turns on and then turns off, and the time at which each white LED 302 of the first illumination device 103 turns on is T=0 (sec). Each white LED 302 of the second illumination device 105 illuminates the other eyeball within the time Δ from this time T=0. The other eyeball thereby receives light stimulation and pupil constriction starts. Then, when each white LED 302 is flashing or lit, the pupil imaging camera 106 continuously captures pupil constriction. That is, the pupil imaging camera 106 captures a plurality of pupil images. The measurement unit 904b then performs image processing on this continuous plurality of pupil images, and thereby obtains the time from the constriction of the pupil starting to completing and calculates the pupil constriction velocity.

In this case, the time Δ depends on the period of time from the time at which pupil constriction of the cow 101 starts to the time at which the cow 101 is startled by the emission of white light onto the eyeball on the one side and runs away. Considering that the light stimulation reaction time is from 0.18 to 0.2 sec for a human, it is desirable that the aforementioned time Δ also be equal to or less than 0.3 sec, and Δ=0 is permissible. In the case where Δ=0, each white LED 302 of the first illumination device 103 emits light at the same time as each white LED 302 of the second illumination device 105.

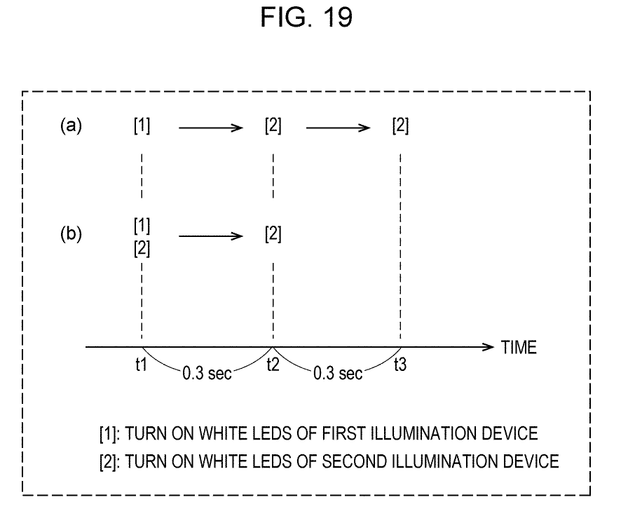

FIG. 19 depicts timings for illumination performed by the first illumination device 103 and the second illumination device 105. It should be noted that, in FIG. 19, [1] indicates a timing at which each white LED 302 (white illumination device) of the first illumination device 103 emits light, and [2] indicates a timing at which each white LED 302 (white illumination device) of the second illumination device 105 emits light.

As depicted in (a) of FIG. 19, each white LED 302 of the second illumination device 105 may emit light at time t2 which is subsequent to time t1 at which each white LED 302 of the first illumination device 103 emitted light, and, in addition, may emit light at time t3 thereafter. Time t2 is a time within 0.3 sec from time t1, and time t3 is a time within 0.3 sec from time t2.

Furthermore, as depicted in (b) of FIG. 19, each white LED 302 of the second illumination device 105 may emit light at time t1 at the same time as each white LED 302 of the first illumination device 103, and may emit light at time t2 thereafter.

### (Estimate Processing Unit 904c)

The estimate processing unit 904c of the estimation unit 904 acquires color information extracted by the extraction unit 904a and a pupil constriction velocity measured by the estimate processing unit 904c, as biological information. The estimate processing unit 904c then estimates the vitamin A blood concentration of the cow 101 by applying the aforementioned acquired biological information to a function indicating the relationship between pre-obtained biological information and the average vitamin A blood concentration of a cow. The estimate processing unit 904c writes the vitamin A blood concentration estimated in this way and the imaging time of the pupil image used for that estimation in the estimate information 902b of the recording unit 902.

### (Notification Unit 905)

The notification unit 905 transmits the image information 902a or the estimate information 902b stored in the recording unit 902 to the mobile terminal 107 in a wireless or wired manner. It should be noted that the mobile terminal 107 is a tablet terminal, a smartphone, a personal computer, or the like of a user such as the fattening farmer.

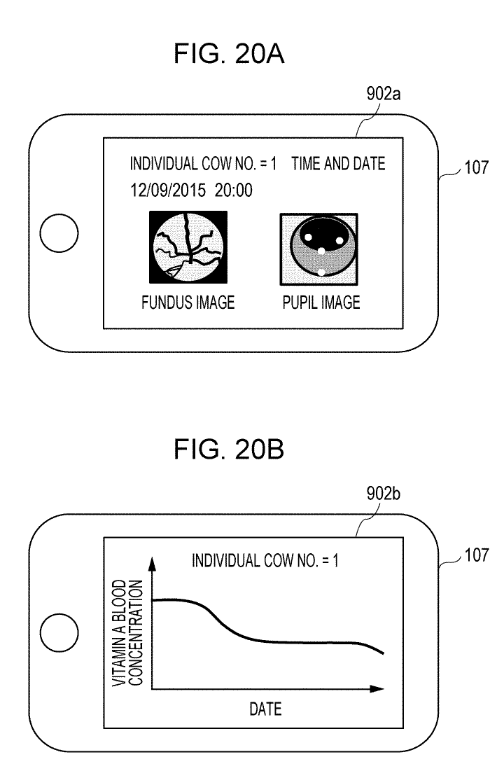

FIG. 20A depicts the image information 902a displayed on the mobile terminal 107.

The notification unit 905 transmits the image information 902a that is read from the recording unit 902, to the mobile terminal 107 of the fattening farmer wirelessly or via a network. The image information 902a is thereby displayed on the display of that mobile terminal 107. As depicted in FIG. 20A, the individual cow No., the imaging time (in other words, the time and date), the fundus image, and the pupil image are displayed on the display. Furthermore, the mobile terminal 107 may receive the individual cow No. and the time and date by means of a user operation performed by the user and transmit such to the notification unit 905, and may acquire and display the image information 902a including the fundus image and pupil image corresponding thereto.

FIG. 20B depicts the estimate information 902b displayed on the mobile terminal 107.

The notification unit 905 transmits the estimate information 902b that is read from the recording unit 902, to the mobile terminal 107 of the fattening farmer wirelessly or via a network. If there are a plurality of items of the estimate information 902b for the same cow 101, the notification unit 905 may transmit the plurality of items of the estimate information 902b.

The estimate information 902b is thereby displayed on the display of that mobile terminal 107. As depicted in FIG. 20B, the individual cow No., the imaging time (in other words, the date), and the vitamin A blood concentration at that imaging time are displayed on the display. In the case where the same individual cow No. is indicated and a plurality of items of the estimate information 902b indicating mutually different imaging times are acquired, the mobile terminal 107 may display the transition in the vitamin A blood concentration of the cow 101 having that individual cow No. together with the time as a graph. Furthermore, the mobile terminal 107 may receive the individual cow No. by means of a user operation performed by the user and transmit such to the notification unit 905, and may acquire and display at least one item of the estimate information 902b corresponding to that individual cow No.

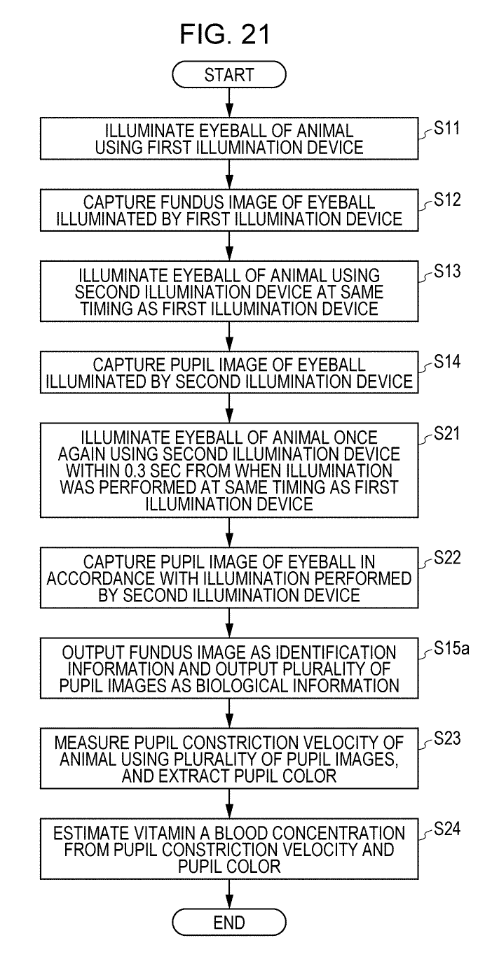

FIG. 21 is a flowchart depicting a processing operation of the camera system 100B in the present embodiment, namely an imaging method for capturing images of the eyeballs of the animal.

The camera system 100B in the present embodiment executes the processing of steps S11 to S14 depicted in FIG. 3 of embodiment 1, and additionally executes the processing of steps S21 to S24 and step S15a.

### (Step S21)

After the processing of steps S11 to S14 has been executed, the second illumination device 105 (specifically, each white LED 302) once again illuminates the eyeball of the cow 101 within 0.3 sec from the point in time of having emitted light in step S13. The point in time of having emitted light in step S13 is the point in time at which the second illumination device 105 emitted light at the same timing as the first illumination device 103 (specifically, each white LED 302).

### (Step S22)

The pupil imaging camera 106 captures a pupil image of the eyeball in accordance with the illumination performed by the second illumination device 105. That is, in steps S14 and S22, the pupil imaging camera 106 captures at least two pupil images.

### (Step S15a)

The output circuit 181 outputs that fundus image to the analysis unit 182 as identification information of the animal, and outputs the plurality of pupil images to the analysis unit 182 as biological information of the animal corresponding to that identification information.

### (Step S23)

The estimation unit 904 of the analysis unit 182, using the plurality of pupil images, measures the pupil constriction velocity of the cow 101, and extracts the pupil color.

### (Step S24)

The estimation unit 904, in addition, estimates the vitamin A blood concentration of the cow 101 from the pupil constriction velocity and the pupil color.

### Effect of Embodiment 2

The camera system 100B in the present embodiment has a configuration similar to that of the camera system 100A of embodiment 1, and therefore demonstrates an effect similar to that of embodiment 1.

Furthermore, the camera system 100B of the present embodiment is additionally provided with an infrared illumination device and the line of sight detection unit 184 that detects the line of sight of the animal. In the case where the first illumination device 103 is configured of a white illumination device (the plurality of white LEDs 302), the aforementioned infrared illumination device is constituted by the plurality of infrared LEDs 303 arranged along the periphery of the fundus imaging camera 104. The fundus imaging camera 104 captures a fundus image for detecting the line of sight of an eyeball illuminated by the infrared illumination device. The line of sight detection unit 184 detects the line of sight of the animal using that fundus image for detecting the line of sight. The first illumination device 103 and the second illumination device 105 illuminate the eyeballs on the basis of that detected line of sight of the animal. The fundus imaging camera 104 captures a fundus image of those eyeballs, and the pupil imaging camera 106 captures a pupil image of those eyeballs.

Specifically, in the present embodiment, the first illumination device 103 and the second illumination device 105 illuminate the eyeballs when the detected line of sight of the animal is the same as the imaging optical axis of the fundus imaging camera 104.

Thus, when the line of sight of that animal is directed toward the fundus imaging camera 104, namely when the pupil of the eyeball is directly facing the fundus imaging camera 104, that eyeball is illuminated by the first illumination device 103, and a fundus image of the illuminated eyeball can be captured. Consequently, a fundus image having a clearer blood vessel pattern depicted therein can be acquired, and highly accurate identification information can be acquired. Furthermore, the second illumination device 105 illuminates the eyeball of the animal at the same timing as the first illumination device 103, and the pupil imaging camera 106 captures a pupil image of that illuminated eyeball. Consequently, it is possible to suppress the line of sight of the animal deviating greatly from the pupil imaging camera 106, namely the pupil of the eyeball not directly facing the pupil imaging camera 106, when the pupil image is captured. As a result, a clear pupil image can be acquired, and highly accurate biological information can be acquired.

Furthermore, in the present embodiment, the measurement unit 904b that measures the pupil constriction velocity of the animal is additionally provided. The second illumination device 105 once again illuminates the eyeball of the animal within 0.3 sec from the point in time of having emitted light at the same timing as the first illumination device 103, and the pupil imaging camera 106 captures a plurality of pupil images in accordance with the illumination performed by the second illumination device 105. The measurement unit 904b measures the pupil constriction velocity of the animal using the plurality of pupil images.

Thus, a highly accurate pupil constriction velocity of the animal can be measured, with reduced effect from pupil constriction or the animal running away due to the eyeballs being illuminated.

### Embodiment 3

In the present embodiment, the individual authentication of the cow 101 is carried out by the photographing of an ear tag by the supplementary individual authentication camera 111 in FIG. 4, or the non-contact reading of a tag by the antenna 112. That is, the camera system in the present embodiment has a configuration similar to that of the camera system 100B of embodiment 2; however, because individual authentication from the fundus image is not carried out, the capturing of a pupil image can be prioritized over the capturing of a fundus image.

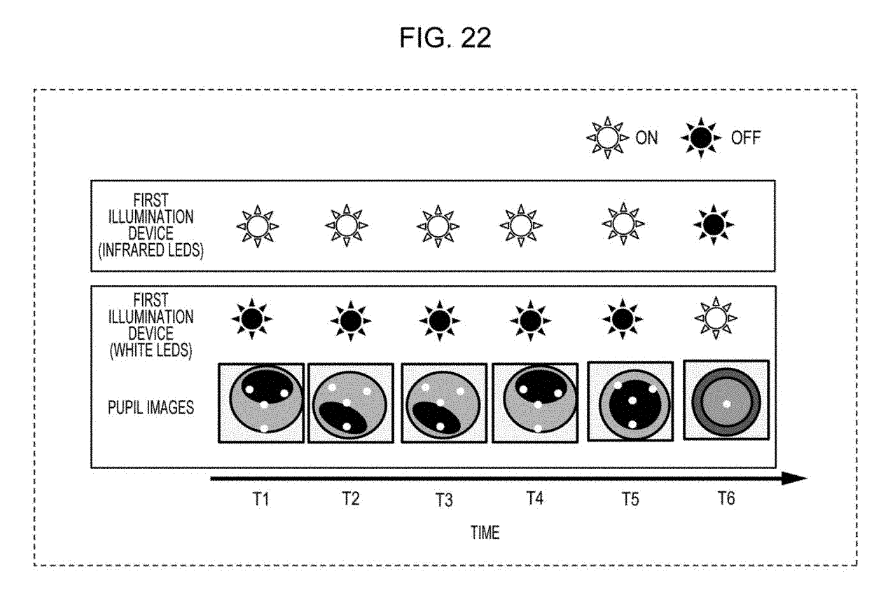

FIG. 22 depicts timings for the illumination performed by the second illumination device 105 and the imaging performed by the pupil imaging camera 106. In embodiment 3, each infrared LED 303 of the second illumination device 105 illuminates an eyeball of the cow 101 without being sensed by the cow 101. At such time, the pupil imaging camera 106 captures a pupil image of the eyeball of the cow 101 illuminated by the infrared light, as an infrared image. The pupil image at such time is also referred to as an infrared pupil image. On the basis of that infrared pupil image, the line of sight detection unit 184 of the analysis control unit 180 continuously detects the line of sight of the eyeball to detect the optimum imaging timing at which the pupil is directly facing the pupil imaging camera 106.

That is, while each infrared LED 303 continuously radiates infrared light onto the eyeball, the pupil imaging camera 106 continuously captures images of the eyeball illuminated by that infrared light, thereby acquiring a plurality of infrared pupil images. The line of sight detection unit 184 tracks the line of sight by continuously carrying out image processing with respect to the plurality of infrared pupil images. The line of sight detection unit 184 then detects the imaging timing at which the pupil is directly facing the pupil imaging camera 106, on the basis of that tracked line of sight. The control unit 183 waits for the imaging timing at which the pupil is directly facing the pupil imaging camera 106. In the example depicted in FIG. 22, the pupil is not directly facing the pupil imaging camera 106 at time slots T1, T2, T3, and T4. The line of sight detection unit 184 detects that the pupil is directly facing the pupil imaging camera 106 at the point in time of time slot T5. As a result, in the next time slot T6, the control unit 183 turns off each infrared LED 303 of the second illumination device 105, and turns on each white LED 302. As a result, the light that illuminates the eyeball of the cow 101 switches from infrared light to white light. In this time slot T6, the pupil imaging camera 106 captures a pupil image of the eyeball illuminated by white light, as a color image.

It should be noted that the fundus imaging camera 104 may capture a fundus image with each white LED 302 of the first illumination device 103 emitting light at the same time as or at a time difference of approximately 0.3 sec from this timing (in other words, time slot T6).

### Embodiment 4

The camera system in the present embodiment carries out individual authentication and the determination of a lesion in real time. This camera system is provided with the constituent elements included in the camera system 100B of embodiment 2 except for the analysis unit 182 and the control unit 183.

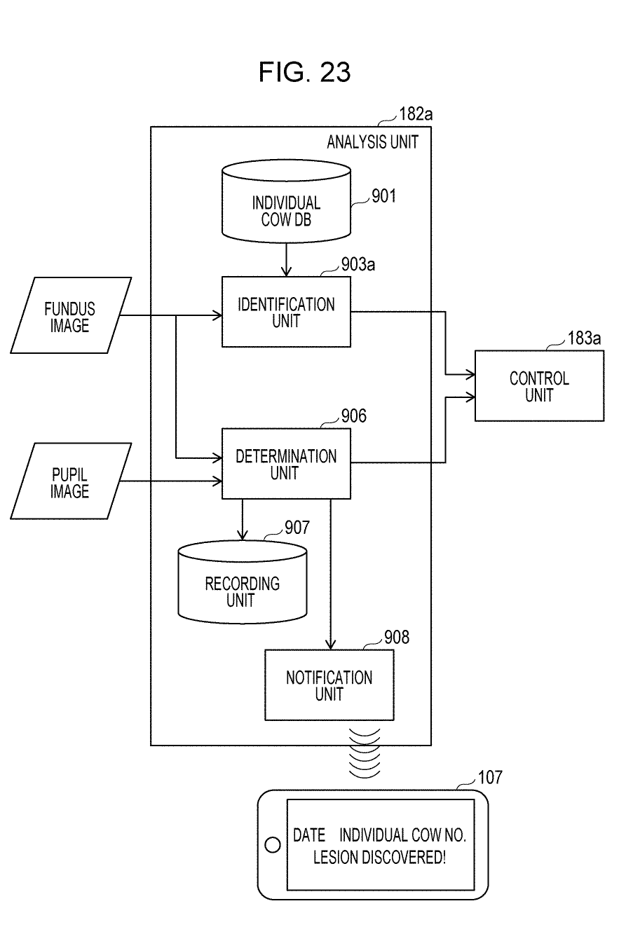

FIG. 23 depicts an analysis unit and a control unit in the present embodiment.

The camera system in the present embodiment is provided with an analysis unit 182a and a control unit 183a instead of the analysis unit 182 and the control unit 183 in embodiment 2.

### (Analysis Unit 182a)

The analysis unit 182a carries out individual authentication and the determination of a lesion in real time, and is provided with the individual cow DB 901, an identification unit 903a, a determination unit 906, a recording unit 907, and a notification unit 908.

### (Identification Unit 903a)

The identification unit 903a, similar to the identification unit 903 of embodiment 2, acquires a fundus image and identifies the individual cow 101 using that fundus image. Reference is made to the identification data in the individual cow DB 901 in this individual identification. The identification unit 903a outputs an individual number indicating the result of that individual identification, to the control unit 183a. Here, the identification unit 903a in the present embodiment identifies the individual cow 101 in real time immediately after a fundus image has been captured by the fundus imaging camera 104. The operations of the illumination performed by the second illumination device 105 and the capturing of the pupil image to be carried out immediately thereafter can be altered as appropriate in accordance with the result of that individual identification. Here, being identified in real time may be that the time from capturing the fundus image to identification is within a time of approximately 0.3 sec.

In addition, the identification unit 903a in the present embodiment determines whether or not individual identification has been successful, each time individual identification is carried out, and in the case where individual identification has failed N times (N being an integer that is equal to or greater than 2), the control unit 183a is notified that identification is not possible.

### (Determination Unit 906)

The determination unit 906 acquires a captured fundus image or a pupil image, and determines in real time whether or not the fundus image or the pupil image includes a lesion. That is, the determination unit 906 diagnoses whether the cow 101 has an illness such as a vitamin A deficiency. For example, similar to embodiment 2, the determination unit 906 determines whether or not the pupil image includes a lesion, according to the pupil color or the pupil constriction velocity. Furthermore, generally, symptoms such as a swelling of the optic nerve head occur in the fundus of a cow having a vitamin A deficiency. Thus, the determination unit 906 determines whether or not there is a lesion on the retina in the fundus image, during individual identification, in other words, in real time. The determination unit 906 outputs the result of that determination to the recording unit 907, the notification unit 908, and the control unit 183a. It should be noted that the determination unit 906 may output information regarding the cow 101 for which a lesion has been determined.

### (Recording Unit 907)

The recording unit 907 records the determination result that is output from the determination unit 906. It should be noted that, in the case where information regarding the cow 101 for which a lesion has been determined is output from the determination unit 906, that information may be recorded in the recording unit 907.

### (Notification Unit 908)

The notification unit 908 acquires the determination result that is output from the determination unit 906, and transmits that determination result to the mobile terminal 107 in a wireless or wired manner. That is, at the same time as a lesion being discovered, the notification unit 908 notifies that discovery of the lesion to the mobile terminal 107 such as a smartphone or a tablet terminal of the fattening farmer.

### (Control Unit 183a)

The control unit 183a acquires notification of the individual number or notification that identification is not possible that is output from the identification unit 903a of the analysis unit 182a, and the determination result that is output from the determination unit 906, and controls the constituent elements of the camera system on the basis of that acquired information.

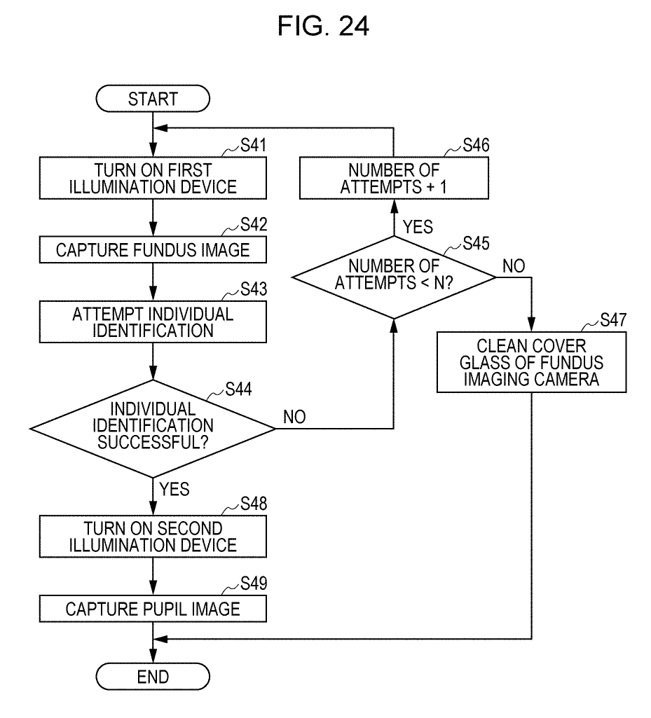

FIG. 24 is a flowchart depicting an example of a control method for the camera system in embodiment 4.

### (Step S41)

The control unit 183a causes the first illumination device 103 to turn on. Specifically, the control unit 183a causes each white LED 302 of the first illumination device 103 to turn on. That is, the first illumination device 103 illuminates an eyeball of the cow 101 with white light.

### (Step S42)

The fundus imaging camera 104 captures a fundus image of the eyeball illuminated by the first illumination device 103.

### (Step S43)

The identification unit 903a attempts individual identification of the cow 101 using the captured fundus image. At such time, the identification unit 903a attempts individual identification in real time.

### (Step S44)

The identification unit 903a determines whether or not individual identification has been successful as a result of that attempt.

### (Step S45)

If it is determined in step S44 that individual identification has not been successful, in other words, has failed (no in step S44), the identification unit 903a additionally determines whether or not the number of attempts at individual identification is less than N times. It should be noted that it is determined that individual identification has failed when the blood vessel pattern of the fundus image does not match the blood vessel pattern on the retina of any of the cows registered in the individual cow DB 901. Furthermore, the initial value for the number of attempts is 1.

### (Step S46)

If it is determined in step S45 that the number of attempts is less than N times (yes in step S45), the identification unit 903a adds 1 to the number of attempts.

### (Step S47)

If it is determined in step S45 that the number of attempts is equal to or greater than N times (no in step S45), the identification unit 903a notifies the control unit 183a that identification is not possible. As a result, the control unit 183a causes the cover glass cleaning device 110 to clean the first cover glass 109a for the fundus imaging camera 104. That is, at such time, because an individual could not be confirmed, the control unit 183a stops the illumination performed by the second illumination device 105 and the imaging performed by the pupil imaging camera 106. The control unit 183a then determines that the first cover glass 109a for the fundus imaging camera 104 is dirty, and causes the cover glass cleaning device 110 to carry out the cleaning of the first cover glass 109a.

### (Step S48)

If it is determined in step S44 that individual identification has been successful (yes in step S44), the identification unit 903a outputs the individual number to the control unit 183a. As a result, the control unit 183a causes the second illumination device 105 to turn on. Specifically, the control unit 183a causes each white LED 302 of the second illumination device 105 to turn on. That is, the second illumination device 105 illuminates the eyeball of the cow 101 with white light.

### (Step S49)

The pupil imaging camera 106 captures a pupil image of the eyeball illuminated by the second illumination device 105.

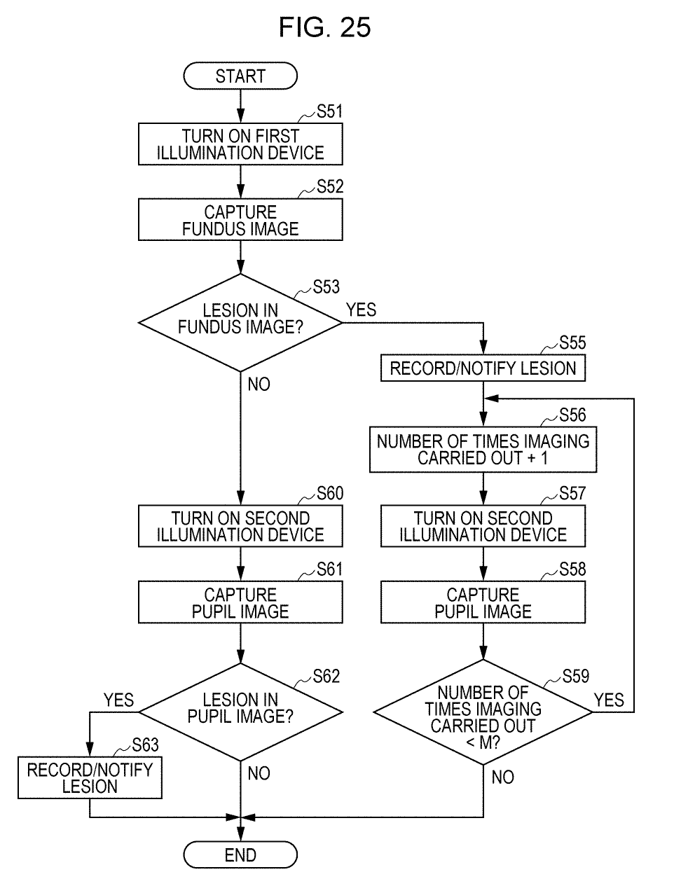

FIG. 25 is a flowchart depicting another example of a control method for the camera system in embodiment 4.

### (Step S51)

The control unit 183a causes the first illumination device 103 to turn on. Specifically, the control unit 183a causes each white LED 302 of the first illumination device 103 to turn on. That is, the first illumination device 103 illuminates an eyeball of the cow 101 with white light.

### (Step S52)

The fundus imaging camera 104 captures a fundus image of the eyeball illuminated by the first illumination device 103.

### (Step S53)

The determination unit 906 acquires a captured fundus image, and determines whether or not the fundus image includes a lesion.

### (Step S55)

If it is determined in step S53 that a lesion is included (yes in step S53), the determination unit 906 records the lesion in the recording unit 907 as the result of that determination. In addition, the notification unit 908 notifies the discovery of the lesion to the mobile terminal 107.

That is, when a fundus image is acquired, the determination unit 906 carries out a lesion diagnosis for a vitamin A deficiency or the like from the fundus image in real time. In the case where a lesion is discovered on the retina in the fundus image when individual identification using the fundus image is carried out, the determination unit 906 determines that the cow 101 corresponding to that fundus image is a cow that has a lesion, and records that lesion in the recording unit 907. The notification unit 908 notifies that lesion to the fattening farmer.

### (Step S56)

The control unit 183a adds 1 to the number of times imaging has been carried out by the pupil imaging camera 106. The initial value for the number of times imaging has been carried out is 0.

### (Step S57)

The control unit 183a causes each white LED 302 of the second illumination device 105 to turn on. That is, the second illumination device 105 illuminates the eyeball of the cow 101 with white light.

### (Step S58)

The pupil imaging camera 106 captures a pupil image of the eyeball illuminated by the second illumination device 105.

### (Step S59)

The control unit 183a determines whether or not the number of times imaging has been carried out is less than M times (M being an integer that is equal to or greater than 2). Here, if it is determined in step S59 that the number of times imaging has been carried out is less than M times (yes in step S59), the control unit 183a repeatedly executes the processing of step S56. However, if it is determined in step S59 that the number of times imaging has been carried out is equal to or greater than M times (no in step S59), the camera system ends processing.

That is, in the case where there is a lesion in the fundus image, the turning on of the second illumination device 105 and the capturing of a fundus image are repeated up to M times in order for observation to be carried out a greater number of times than normal.

### (Step S60)

If it is determined in step S53 that the fundus image does not include a lesion (no in step S53), the control unit 183a causes each white LED 302 of the second illumination device 105 to turn on. That is, the second illumination device 105 illuminates the eyeball of the cow 101 with white light.

### (Step S61)

The pupil imaging camera 106 captures a pupil image of the eyeball illuminated by the second illumination device 105.

### (Step S62)

The determination unit 906 acquires the captured pupil image, and determines whether or not the pupil image includes a lesion. Here, if it is determined that a lesion is not included (no in step S62), the camera system ends processing.

### (Step S63)

If it is determined in step S62 that a lesion is included (yes in step S62), the determination unit 906 records the lesion in the recording unit 907 as the result of that determination. In addition, the notification unit 908 notifies the discovery of the lesion to the mobile terminal 107.

In this way, a lesion is recorded in the recording unit 907 and notified to the fattening farmer in the case where a lesion is not found in the fundus image but it is then determined in real time that there is a lesion from observation of the pupil image. Furthermore, the light emission pattern of the plurality of white LEDs 302 may be changed or the number of times imaging is carried out may be increased in such a way that it is possible for a detailed observation to be carried out at the next imaging timing.

Furthermore, in the flowchart depicted in FIG. 25, if it is determined in step S53 that a lesion is included, the animal is illuminated by the second illumination device 105; however, the animal may not be illuminated by the second illumination device 105. It is thereby possible to prevent going to the trouble of capturing a pupil image in order to determine whether or not there is a lesion, also in the case where it can be determined from a fundus image that there is a lesion in an animal.

### Effect of Embodiment 4

The camera system in the present embodiment has a configuration similar to that of the camera system 100A of embodiment 1, and therefore demonstrates an effect similar to that of embodiment 1.

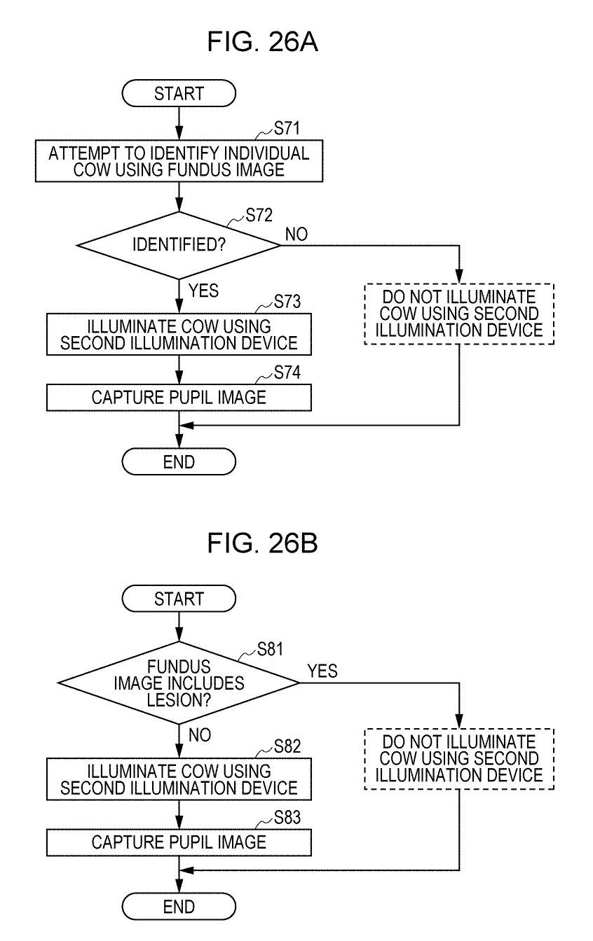

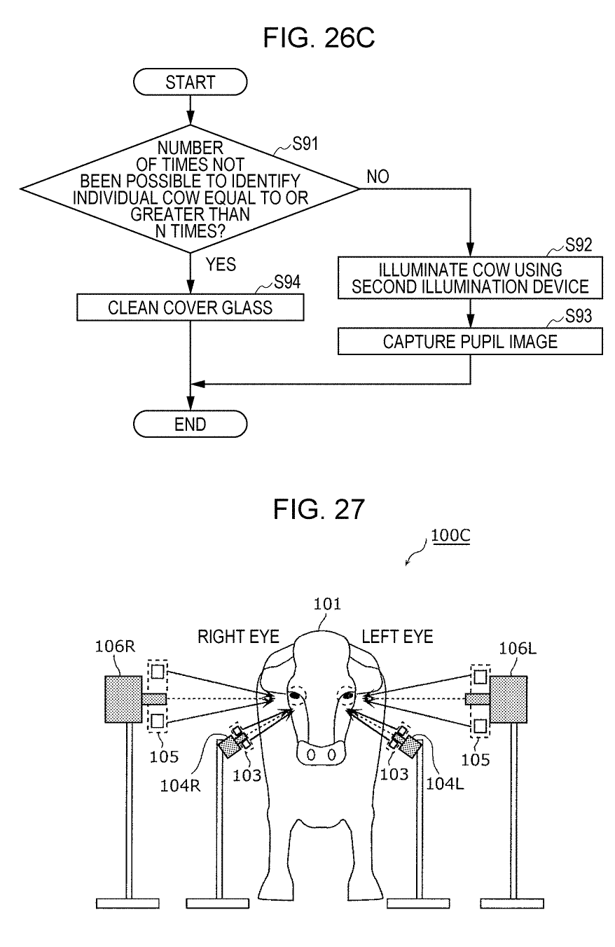

Furthermore, in the present embodiment, as mentioned above, whether or not illumination is to be carried out by the second illumination device 105 and whether or not cleaning is to be carried out by the cover glass cleaning device 110 is controlled. A summary of this kind of control and the effect thereof are described hereinafter using FIGS. 26A to 26C.

FIG. 26A is a flowchart depicting an example of the control of the second illumination device 105 and the pupil imaging camera 106 performed by the control unit 183a in the present embodiment. It should be noted that this flowchart includes processing corresponding to steps S43, S44, S48, and S49 of the flowchart in FIG. 24.

### (Step S71)

The identification unit 903a attempts to identify the individual cow 101 using the fundus image in accordance with the control carried out by the control unit 183a. That is, the identification unit 903a attempts individual identification of the cow 101.

### (Step S72)

The control unit 183a determines whether or not the identification unit 903a has been able to identify the individual cow 101. Here, if the identification unit 903a has not been able to identify the individual cow 101 (no in step S72), the control unit 183a does not illuminate the cow 101 by means of the second illumination device 105.

### (Step S73)

However, in step S72, if it is determined that the identification unit 903a has been able to identify the individual cow 101 (yes in step S72), the control unit 183a illuminates the cow 101 by means of the second illumination device 105. That is, the control unit 183a causes each white LED 302 of the second illumination device 105 to turn on.

### (Step S74)

The pupil imaging camera 106 captures a pupil image of the eyeball illuminated by the second illumination device 105, in accordance with the control carried out by the control unit 183a.

Thus, in the present embodiment, a pupil image being acquired as biological information can be prevented until it is not possible to identify the animal, and wasteful processing and the accumulation of information can be eliminated.

FIG. 26B is a flowchart depicting another example of the control of the second illumination device 105 and the pupil imaging camera 106 performed by the control unit 183a in the present embodiment. It should be noted that this flowchart includes processing corresponding to steps S53, S60, and S61 of the flowchart in FIG. 25.

### (Step S81)

The determination unit 906 determines whether or not the fundus image includes a lesion. Here, if it is determined that a lesion is included (yes in step S81), the control unit 183a does not illuminate the cow 101 by means of the second illumination device 105.

### (Step S82)

However, if it is determined in step S81 that a lesion is not included (no in step S82), the control unit 183a illuminates the cow 101 by means of the second illumination device 105. That is, the control unit 183a causes each white LED 302 of the second illumination device 105 to turn on.

### (Step S83)

The pupil imaging camera 106 captures a pupil image of the eyeball illuminated by the second illumination device 105, in accordance with the control carried out by the control unit 183a.

Thus, in the present embodiment, it is possible to prevent going to the trouble of capturing a pupil image in order to determine whether or not there is a lesion, also in the case where it can be determined from a fundus image that there is a lesion in an animal. It is thereby possible to eliminate wasteful processing and the accumulation of information.

FIG. 26C is a flowchart depicting another example of the control of the second illumination device 105 and the pupil imaging camera 106 performed by the control unit 183a in the present embodiment. It should be noted that this flowchart includes processing corresponding to steps S44, S45, and S47 to S49 of the flowchart in FIG. 24.

### (Step S91)

The control unit 183a determines whether or not the number of times it has not been possible to identify the individual cow 101 is equal to or greater than a predetermined number of times (for example, N times), on the basis of the results of the individual identification of the cow 101 repeatedly attempted by the identification unit 903a.

### (Step S92)

If the number of times it has not been possible to identify the individual cow 101 is not equal to or greater than the predetermined number of times (no in step S91), the control unit 183a illuminates the cow 101 by means of the second illumination device 105. That is, the control unit 183a causes each white LED 302 of the second illumination device 105 to turn on.

### (Step S93)

The pupil imaging camera 106 captures a pupil image of the eyeball illuminated by the second illumination device 105, in accordance with the control carried out by the control unit 183a.

### (Step S94)

If the number of times it has not been possible to identify the individual cow 101 is equal to or greater than the predetermined number of times (yes in step S91), the control unit 183a causes the cover glass cleaning device 110 to clean the first cover glass 109a for the fundus imaging camera 104. This first cover glass 109a is glass that covers the fundus imaging camera 104, between the fundus imaging camera 104 and the cow 101.

Thus, in the present embodiment, in the case where the identification of the individual animal fails a predetermined number of times or more, because the first cover glass 109a is cleaned, it is possible to suppress the failure of the individual identification after the first cover glass 109a has been cleaned.

### Embodiment 5

The camera system in the present embodiment has a configuration in which a fundus imaging camera and a pupil imaging camera are installed with respect to each of the two eyeballs of the cow (what is known as a single lens multi-camera configuration).

FIG. 27 depicts the camera system in the present embodiment.

A camera system 100C in the present embodiment is provided with a fundus imaging camera 104R and a pupil imaging camera 106R that capture images of the right eye of the cow 101, and a fundus imaging camera 104L and a pupil imaging camera 106L that capture images of the left eye of the cow 101.

The fundus imaging camera 104R and the fundus imaging camera 104L have the same configuration as the fundus imaging camera 104 in the aforementioned embodiments. The pupil imaging camera 106R and the pupil imaging camera 106L have the same configuration as the pupil imaging camera 106 in the aforementioned embodiments. Furthermore, similar to the aforementioned embodiments, the first illumination device 103 is arranged in the fundus imaging camera 104R and the fundus imaging camera 104L. Likewise, similar to the aforementioned embodiments, the second illumination device 105 is arranged in the pupil imaging camera 106R and the pupil imaging camera 106L.

It should be noted that, similar to the camera system of any of embodiments 1 to 4, the camera system in the present embodiment may not be provided with the output circuit 181, the analysis control unit 180, the individual authentication camera 111, or the antenna 112 for RFID.

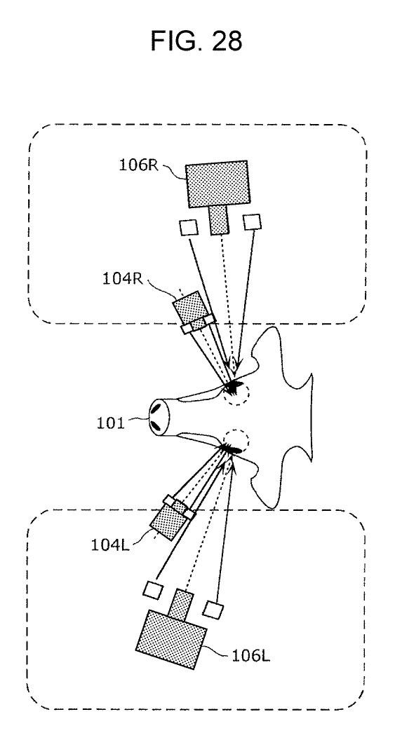

FIG. 28 is a drawing in which the camera system 100C is seen from above. The fundus imaging cameras 104R and 104L are each installed in positions that are closer in terms of distance than the pupil imaging cameras 106R and 106L, and capture fundus images of the eyeballs. In the capturing of these fundus images, the fundus imaging cameras 104R and 104L each capture fundus images of the eyeballs illuminated by the first illumination devices 103 at an illuminance greater than the illuminance of the illumination performed by the second illumination devices 105.

According to this configuration, for example, individual identification using the left eye can be carried out even in the case where it is established in real time that individual identification has failed due to any kind of cause with the fundus imaging camera 104R for the right eye. That is, immediately after that failure has been established, individual identification can be carried out by means of the imaging performed by the fundus imaging camera 104L for the left eye, and the capturing of a pupil image performed by the pupil imaging camera 106R for the right eye can be carried out immediately thereafter. Similarly, even in the case where individual identification using the fundus image captured by the fundus imaging camera 104R for the right eye has failed when a lesion has been discovered in that fundus image, individual identification can be carried out by means of the imaging performed by the fundus imaging camera 104 for the left eye. In this way, it becomes possible for the roles of the cameras to be exchanged in a short period of time.

### Embodiment 6

The system in the present embodiment is a feeding system that feeds an animal using a fundus image and a pupil image of that animal captured by a camera system.

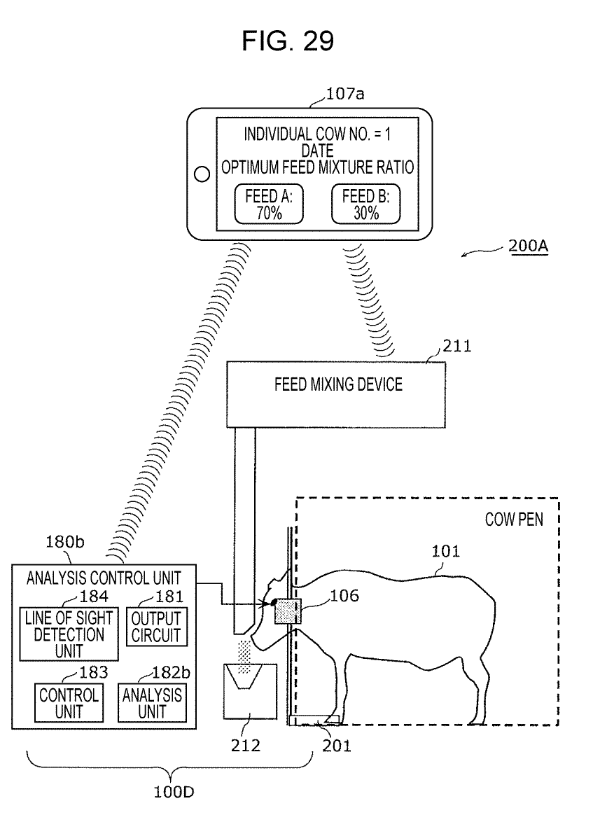

FIG. 29 depicts an example of a configuration of the feeding system in the present embodiment A feeding system 200A depicted in FIG. 29 feeds an animal using a fundus image and a pupil image of that animal captured by a camera system 100D.

This feeding system 200A is provided with the camera system 100D, a mobile terminal 107a, and a feed mixing device 211. It should be noted that constituent elements that are the same as any of those of embodiments 1 to 5 from among the constituent elements included in the feeding system 200A in the present embodiment are denoted by the same reference numerals and detailed descriptions thereof are omitted.

### (Mobile Terminal 107a)

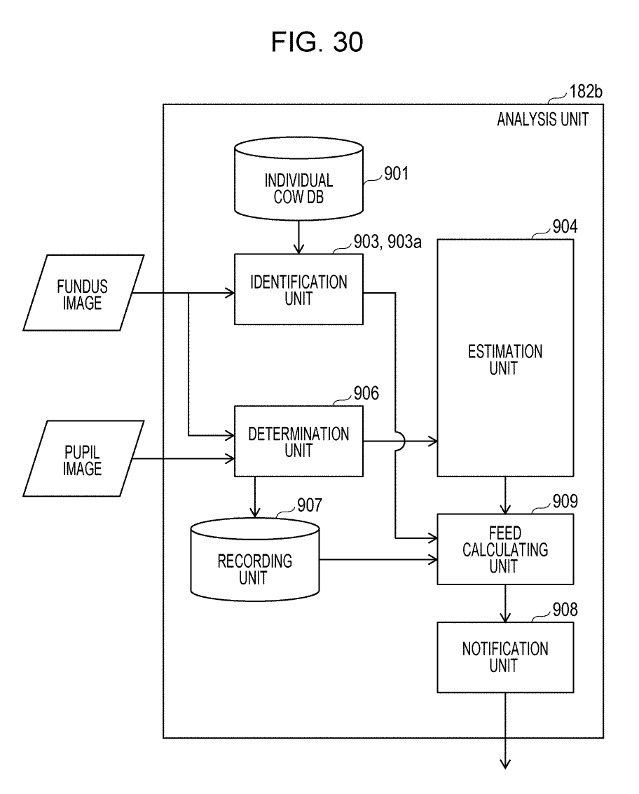

The mobile terminal 107a is an interface that outputs a signal for switching the composition of the feed, corresponding to the concentration of vitamin A estimated by the camera system 100D. It should be noted that the concentration of vitamin A estimated by the camera system 100D is the concentration of vitamin A estimated by the estimation unit 904 (see FIG. 30), which is described later on, provided in the camera system 100D. That is, the mobile terminal 107a is an interface between the user and the feeding system 200A, and acquires information in a wireless or wired manner from the camera system 100D and displays that information. That information is the optimum feed composition ratio or the like for the cow 101, calculated using the concentration of vitamin A in the blood of the cow 101 estimated by the camera system 100D. Furthermore, the mobile terminal 107a receives an operation from the user, and outputs a signal for switching the composition of the feed to that optimum feed composition ratio, to the feed mixing device 211 in a wireless or wired manner. The feeding system 200A in the present embodiment is provided with the mobile terminal 107a as an example of an interface; however, it should be noted that the feeding system 200A may be provided with another apparatus, device, or the like as an interface. For example, the interface may be an input device, a display, a tablet terminal, a smartphone, a personal computer, or the like. An input device, for example, is a keyboard, a mouse, a touch panel, or the like.

### (Feed Mixing Device 211)

The feed mixing device 211, upon receiving the aforementioned signal from the mobile terminal 107a, switches the composition of feed that enters a feed trough 212 to the optimum feed composition ratio indicated by that signal.

### (Camera System 100D)

The camera system 100D, similar to embodiment 2, is provided with the first illumination device 103, the fundus imaging camera 104, the second illumination device 105, and the pupil imaging camera 106, and is additionally provided with an analysis control unit 180b. It should be noted that FIG. 29 depicts the pupil imaging camera 106 and the analysis control unit 180b from among the constituent elements included in the camera system 100D.

The first illumination device 103 illuminates an eyeball of the cow 101. The fundus imaging camera 104 captures a fundus image of the eyeball illuminated by the first illumination device 103. The second illumination device 105 illuminates an eyeball of the animal at the same timing as the first illumination device 103. The pupil imaging camera 106 captures a pupil image of the eyeball illuminated by the second illumination device 105.

### (Analysis Control Unit 180b)

The analysis control unit 180b, similar to embodiment 2, is provided with the output circuit 181, the control unit 183, and the line of sight detection unit 184, and is additionally provided with an analysis unit 182b.

The output circuit 181 outputs the fundus image as identification information of the cow 101, and outputs the pupil image as biological information of the cow 101 corresponding to that identification information. Specifically, the output circuit 181 outputs the identification information and the biological information to the analysis unit 182b.

### (Analysis Unit 182b)

The analysis unit 182b estimates the concentration of vitamin A in the blood of the cow 101 using the pupil image, and calculates the optimum feed composition ratio for the cow 101 using that estimated vitamin A concentration. The analysis unit 182b then notifies information indicating that optimum feed composition ratio to the mobile terminal 107a.

FIG. 30 is a block diagram of the analysis unit 182b.

The analysis unit 182b, similar to embodiments 2 and 4, is provided with the individual cow DB 901, the identification unit 903, the estimation unit 904, the recording unit 907, and the notification unit 908, and is additionally provided with a feed calculating unit 909. It should be noted that the analysis unit 182b may be provided with the identification unit 903a instead of the identification unit 903. The estimation unit 904, similar to embodiment 2, estimates the concentration of vitamin A in the blood of the cow 101 using the pupil image.

### (Feed Calculating Unit 909)

The feed calculating unit 909 calculates the optimum feed composition ratio for the cow 101 using the vitamin A concentration estimated by the estimation unit 904. Furthermore, the feed calculating unit 909 calculates a feed composition ratio with which the vitamin A in the blood is maintained while preventing the blindness or illness of the cow 101, from the current vitamin A blood concentration estimated by the estimation unit 904, past vitamin A blood concentrations, and clinical history records. In addition, the feed calculating unit 909 outputs that information indicating the feed composition ratio to the notification unit 908. For example, the feed calculating unit 909 retains a function or table that indicates the correlation between the concentration of vitamin A in the blood and the ratio of feed A with respect to the total feed, and derives the ratio of feed A corresponding to the current estimated concentration of vitamin A in the blood from that function or table. The optimum feed composition ratio is thereby calculated. Furthermore, the feed calculating unit 909 may calculate the difference between a concentration of vitamin A in the blood estimated in the past and the current concentration of vitamin A in the blood, and may apply a coefficient corresponding to that difference to that derived feed A ratio. It is thereby possible to also handle sudden changes in the concentration of vitamin A in the blood. Furthermore, when the feed calculating unit 909 refers to lesion records, specifies the ratio of feed A to be given to the cow 101 when a lesion has appeared, and derives the ratio of feed A from the aforementioned function or table, the feed calculating unit 909 may carry out the derivation avoiding feed A ratios from when a lesion has appeared.

The notification unit 908 in the present embodiment notifies the feed composition ratio calculated by the feed calculating unit 909 to the mobile terminal 107a.

Thus, the information (specifically, the information indicating the feed composition ratio) notified from the notification unit 908 is displayed on the display of the mobile terminal 107a such as a smartphone or a tablet terminal of the fattening farmer who is the user, as depicted in FIG. 29. The user transmits an instruction regarding the optimum feed composition ratio for the specific cow 101 to the feed mixing device 211 using an interface constituted by that mobile terminal 107a. The feed mixing device 211 then sets the optimally mixed feed to the feed trough 212 specifically for that cow 101 in the cow pens. It should be noted that the cow 101 is able to eat the feed from this feed trough 212 only when individual identification of that cow 101 has been carried out. This individual identification may be carried out on the basis of the imaging performed by the fundus imaging camera 104, or may be carried out by means of the photographing of an ear tag performed by the supplementary individual authentication camera 111 in FIG. 4 of embodiment 2 or by means of the non-contact reading of a tag performed by the antenna 112.

Furthermore, the mobile terminal 107a may receive the individual cow No. by means of a user operation performed by the user and transmit such to the notification unit 908. In this case, the notification unit 908 notifies the mobile terminal 107a of the most up-to-date feed composition ratio calculated by the feed calculating unit 909 for the cow 101 identified by means of that individual cow No. The mobile terminal 107a then displays an image depicting that individual cow No. and the feed composition ratio, on a display as depicted in FIG. 29.

### Effect of Embodiment 6

The feeding system 200A in the present embodiment feeds an animal using a fundus image and a pupil image of the animal captured by the camera system 100D. The camera system 100D is provided with the first illumination device 103, the fundus imaging camera 104, the second illumination device 105, the pupil imaging camera 106, the output circuit 181, the estimation unit 904, and the mobile terminal 107a. The first illumination device 103 illuminates an eyeball of the animal. The fundus imaging camera 104 captures a fundus image of the eyeball illuminated by the first illumination device 103. The second illumination device 105 illuminates an eyeball of the animal at the same timing as the first illumination device 103. The pupil imaging camera 106 captures a pupil image of the eyeball illuminated by the second illumination device 105. The output circuit 181 outputs the fundus image as identification information of the animal, and outputs the pupil image as biological information of the animal corresponding to that identification information. The estimation unit 904 estimates the concentration of vitamin A in the blood of the animal using that pupil image. The mobile terminal 107a is an interface that outputs a signal for switching the composition of the feed, corresponding to the concentration of vitamin A estimated by the estimation unit 904.

This kind of feeding system 200A or camera system 100D in the present embodiment has a configuration similar to that of the camera system 100A of embodiment 1, and therefore demonstrates an effect similar to that of embodiment 1.

Furthermore, in the present embodiment, the vitamin A blood concentration of an animal can be acquired while that individual animal is appropriately identified, and feed to be given to that animal can be made to have the optimum feed composition ratio corresponding to the vitamin A blood concentration of that animal. For example, the cow 101 can be fed with the optimum feed composition ratio for improving the meat quality without a severe illness such as blindness occurring.

### Embodiment 7

In embodiment 7, the main purpose is mainly to capture a pupil image of a cow with a high degree of quality. Ordinarily, in the non-contact acquisition of a pupil, the eyeball of a cow is not always positioned in the center of a screen and is often captured deviating randomly to the left and right of the screen. In addition, the line of sight of an eyeball does not face the front of the imaging optical axis but deviates diagonally upward or diagonally downward, and therefore the pupil is captured close to an ellipse rather than a true circle. This is due not to an error of a sensor that decides the imaging timing but to it being not possible in reality for the direction of the line of sight of an eyeball at the imaging timing to be fixed. In an imaging method such as this, the position and angle at which light that is incident on the pupil is radiated onto the retina is not fixed, and the angle of outgoing light from the pupil changes in numerous ways with respect to the line of sight of the camera, and therefore, in the case where the color of the reflected light from the tapetum layer of the retina is reflected as the pupil color, that pupil color generally changes in numerous ways. In this way, the line of sight of an eyeball of a cow cannot be fixed, and therefore the color of the tapetum layer cannot be measured as the pupil color with a high degree of accuracy from outside, and consequently there is a problem in that there is a decline in the accuracy of estimating the vitamin A concentration.

Even in the case where imaging can be performed with the line of sight of the eyeball matching the illumination and imaging optical axes, the pupil color is not one complete color, and a color irregularity occurs with reflected light of a blue-green color from the tapetum region and dark red reflected light from the non-tapetum region being present according to the region. It is therefore difficult to observe the color of the tapetum region.

The present embodiment solves the aforementioned problems, and a purpose thereof is to provide an animal eye imaging device that can acquire a reflected color from the tapetum with a sufficiently high degree of accuracy even in the non-contact observation of the pupil color.

In order to implement a state in which the line of sight is fixed to the front when seen from a camera, the eye of the cow may be continuously observed with invisible infrared illumination being emitted toward the cow, and color imaging may be carried out with white illumination being radiated in a stroboscopic manner at a timing at which the line of sight is matching. However, when carried out using one imaging device for each eye as in the prior art, there are very few imaging chances. Thus, a plurality (nine, for example) of viewpoint cameras, which have white light sources attached thereto in a substantially coaxial state, and infrared light sources are installed for an eyeball to be observed in the same manner from a plurality of viewpoints by means of infrared illumination, and white light is radiated from the white light source corresponding to the viewpoint camera that matches the line of sight for color imaging to be carried out. A pupil image in which the line of sight matches as much as possible can thereby be acquired without causing unnecessary stress such as forcibly guiding the line of sight of an eyeball of the cow.

Next, regarding the problem that there is a color irregularity, in other words, the regions of tapetum-region reflected color (yellow to green to blue) and a non-tapetum region (red eye), within a pupil even when the line of sight and optical axis match, there is a problem in that separation with a color filter is not possible because the tapetum color spectrum is wide (a wide range of 400 to 700 nm). Thus, the fact that the reflected light from the tapetum region is similar to mirror surface reflection is used, polarized illumination is radiated to generate a “parallel” and “orthogonal” difference polarized image S, and a non-polarized (tapetum) region is eliminated, in other words, values are set to 0 (to black) on an image for the tapetum region to be extracted.

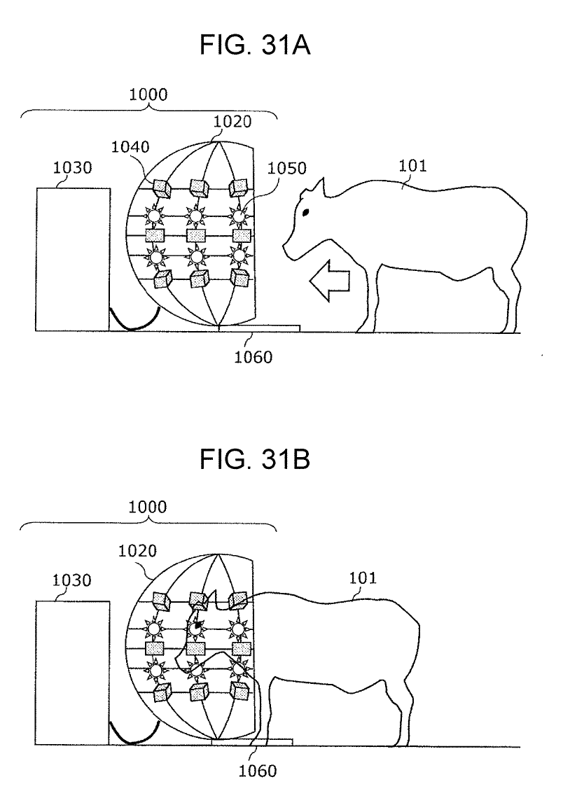

FIGS. 31A and 31B depict drawings in which an animal eye imaging device 1000 according to embodiment 7 is seen from the side. The animal eye imaging device 1000 is configured of an imaging dome 1020 and a control unit 1030. The imaging dome 1020 is substantially hemispherical and may be formed by means of a structure such as a frame or a transparent body. The animal eye imaging device is installed adjacent to the cattle barn in FIG. 31A. An opening of a sufficient size into which the cow 101 is able to approach from the cattle barn and insert its head is provided, and a water drinking station, which is not depicted, is installed near the center. A plurality of white light source-equipped color cameras 1040 and infrared light sources 1050 are installed in the imaging dome in such a way that images of the left and right eyeballs of the cow can be captured from a plurality of viewpoints. In FIG. 31A, the cow 101 is approaching the water drinking station in the center of the imaging dome from inside the cattle barn at night. In FIG. 31B, the cow 101 has entered inside the imaging dome and is drinking water, and this state is detected by a pressure sensor 1060. The plurality of white light source-equipped color cameras 1040 and the infrared light sources 1050 installed in the imaging dome 1020 operate during this water intake period of the cow in accordance with an instruction from the control unit 1030 to capture color images of the pupils of the left and right eyeballs of the cow. These images are image-processed and recorded in the control unit 1030. In this way, in the animal eye imaging device 1000, the health condition of the cow is recorded with the acquisition of the pupil image, which is conventionally carried out with an imaging device being pressed up against an eyeball of the cow by a livestock raiser or a veterinarian, being carried out at night completely automatically in a non-contact manner without the cow being touched at all. At the same time, the individual identification of the cow may be carried out by means of a technology such as image sensing or an RFID tag, and may be recorded together with the pupil image.

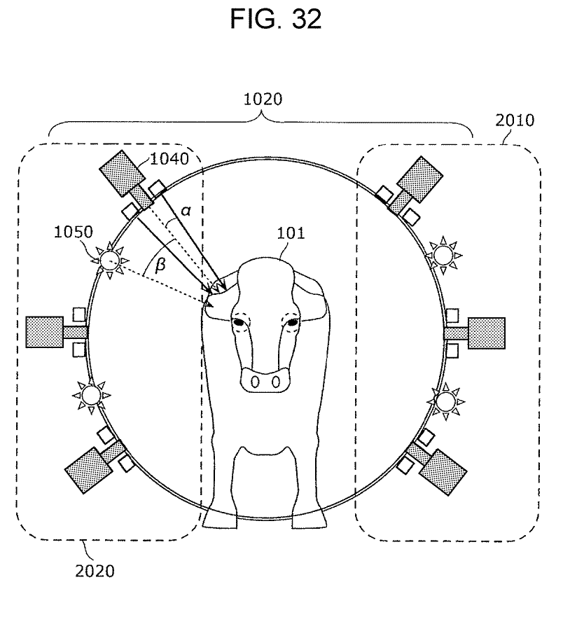

FIG. 32 depicts a drawing in which the animal eye imaging device 1000 according to embodiment 7 is seen from the front. A plurality of the white light source-equipped color cameras 1040 and infrared light sources 1050 installed at the same longitude in the imaging dome are depicted. The white light source optical axes and the imaging optical axes of the white light source-equipped color cameras 1040 have a substantially coaxial relationship. “Substantially coaxial” indicates that an angle α formed by both optical axes is sufficiently small, and an image of the pupil can be captured with a good degree of brightness at this angle. In a pupil image, when a color camera is used, a tapetum is not present on the retina in the case of a human and reflected light from blood vessels becomes return light, resulting in what is known as “red eye”. However, with an animal having a tapetum such as a cow, colored light from the tapetum which has an extremely high reflectance is reflected, and therefore observing the pupil color is substantially the same as observing the tapetum colors as they are, and it is therefore possible for a tapetum color of a blue-green color to be measured with a high degree of accuracy from outside even without dissecting the eyeball.

Meanwhile, although a plurality of infrared light sources 1050 are installed inside the imaging dome, they do not have a substantially coaxial relationship with the imaging devices, and with infrared illumination, the reflected light from the pupil is captured in monochrome as black whereas the surrounding iris and the skin of the cow are captured as white with a high degree of luminance, and therefore there is a feature in that it becomes extremely easy to detect the pupil with contrast. Using this it is possible to determine by means of image processing whether the line of sight of an eyeball is directly facing or deviating from the imaging optical axis.

The white light source-equipped color cameras 1040 are divided in two into a group 2010 for the left eye and a group 2020 for the right eye of the cow, and the cameras of the respective groups capture images of the corresponding left or right eyeball.

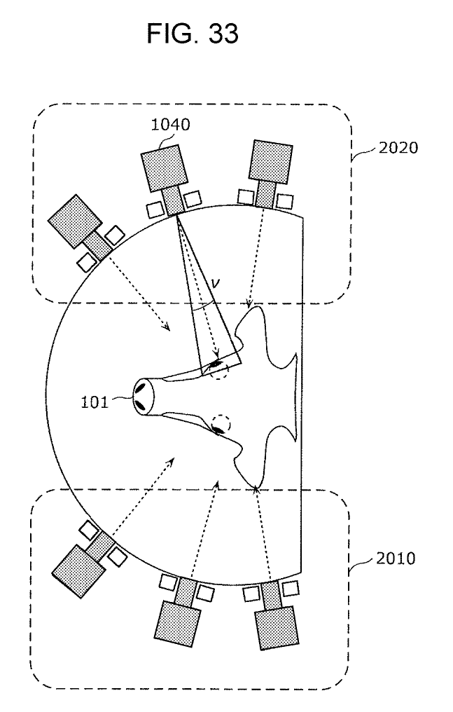

FIG. 33 depicts a drawing in which the animal eye imaging device 1000 according to embodiment 7 is seen from above. A plurality of the white light source-equipped color cameras 1040 installed at the same latitude in the imaging dome are depicted. In the case of a cow, the left and right eyeballs are positioned on both sides of the head, and therefore the white light source-equipped color cameras 1040 are installed concentrated in the left and right hemispheres of the imaging dome, and, as previously mentioned, the cameras of the group 2010 for the left eye and the group 2020 for the right eye of the cow capture images of the eyeball to which the cameras of the respective groups correspond. The viewing angle γ of the color cameras is the smallest angle at which the whole of the surrounding eye can be captured with the pupil of the cow at the center, enabling a pupil image to be captured as large as possible.

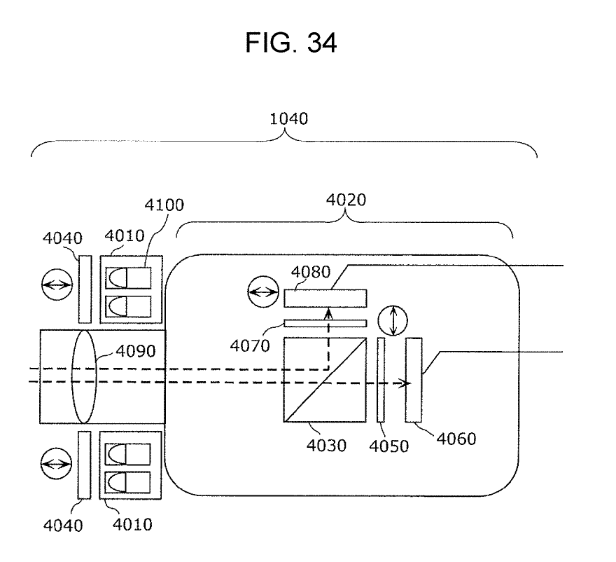

FIG. 34 is a drawing describing a configuration of the white light source-equipped color cameras 1040, configured of a white ring illumination device 4010, a color polarization camera 4020, a lens unit 4090, and a donut-shaped polarizing plate 4040. The white ring illumination device is configured of a set of white light source LEDs 4100, and the transmission optical axis of the polarizing plate is set here to be horizontal (H), and therefore white polarized illumination having a horizontal axis can be captured. In this embodiment, the color polarization camera 4020 is configured of a beam splitter 4030, polarizing plates 4050 and 4070, and single-plate color imaging elements 4060 and 4080. The return light that is incident on the camera is divided into two optical paths by the beam splitter 4030, with the return light being transmitted through the horizontal (H)-axis polarizing plate 4070 and converted into an image by the single-plate color imaging element 4080 and output as a parallel polarized image, and being transmitted through the vertical (V)-axis polarizing plate 4050 and converted into an image by the single-plate color imaging element 4060 and output as a vertical polarized image. In this way, the color polarization camera 4020 is able to simultaneously capture and output two color images having polarization axes that are parallel and orthogonal with respect to the polarization axis of the polarized illumination.

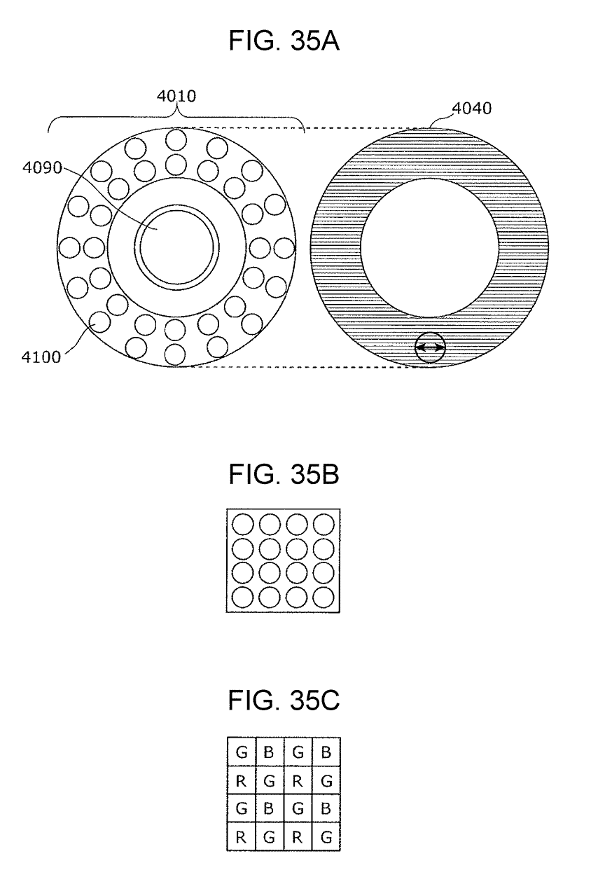

FIGS. 35A to 35C are drawings depicting details of illumination and an imaging element. FIG. 35A depicts the white ring illumination device 4010 which is configured of a plurality of rows of white LEDs, and the donut-shaped polarizing plate 4040 which has a horizontal (H) transmission axis. The white LEDs may be close to a natural light spectrum with a visible light range of approximately 400 to 800 nm. FIG. 35B depicts a configuration of an infrared light source, in which infrared LEDs in the vicinity of 850 nm are configured as a surface light source. FIG. 35C depicts a portion of the configuration of the single-plate color imaging elements 4060 and 4080, in which an ordinary Bayer color mosaic filter is used.

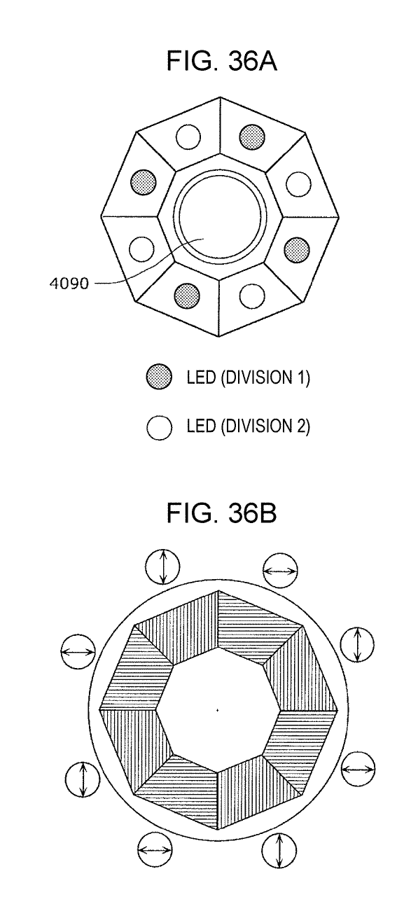

FIGS. 36A and 36B are drawings depicting another configuration for polarized illumination. In these drawings, the differences with FIG. 25A are that independent light emission is possible with the white LEDs being divided into two channels as indicated by “division 1” and “division 2”, and that polarizing plates having horizontal (H) transmission axes are installed for the LEDs of division 1 and polarizing plates having vertical (V) transmission axes are installed for the LEDs of division 2. In the case where a difference polarized image described later on is calculated by combining this illumination device and the color polarization camera 4020 depicted in FIG. 34, it becomes possible to observe the two types of a pair of a parallel polarized image and an orthogonal polarized image, and a difference polarized image having low noise can be obtained with a high degree of accuracy.

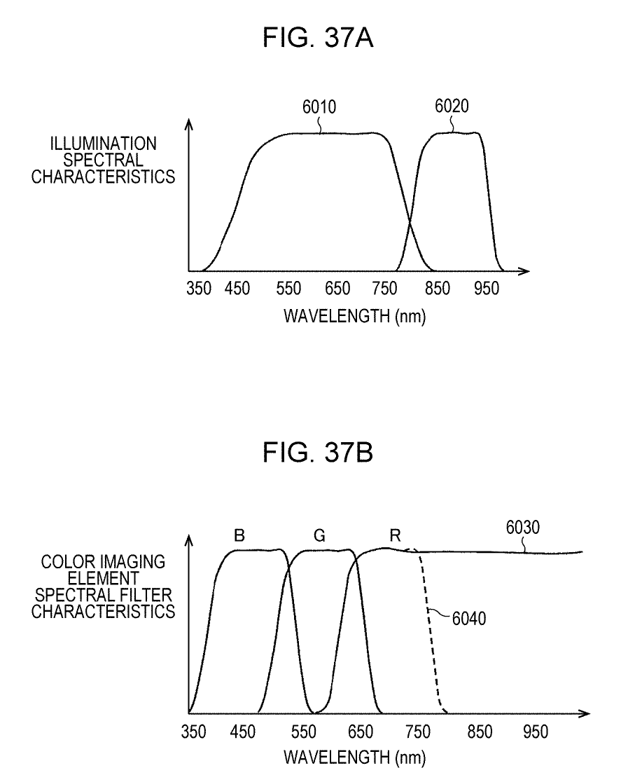

FIGS. 37A and 37B are drawings depicting spectral distributions of light sources and imaging. FIG. 37A is a drawing depicting a spectral energy distribution of a white light source and an infrared light source. As previously mentioned, a natural light white LED in which the spectral distribution of the white light source has broad characteristics in the visible region may be used. An example of the characteristics of the spectral distribution of a natural light white LED is indicated by 6010. The infrared light source has a spectral distribution centered about the vicinity of 850 nm as indicated by 6020.

FIG. 37B is a drawing describing the spectral sensitivity of the single-plate color imaging elements 4060 and 4080. The spectral sensitivities for B (blue) and G (green) are as normal. For R (red), however, an IR cut filter is not used in order to acquire an infrared image. Therefore, the spectral characteristics do not have a normal shape such as that indicated by 6040 and correspond to the spectral distribution 6020 of an infrared light source as indicated by 6030. According to this configuration, in a period during which only a white light source is lit, the subject is illuminated by a white light source distribution and therefore normal RGB color imaging becomes possible with the 6030 portion being cut, and in a period during which only an infrared light source is lit, an R (red) image performs the role of a monochrome infrared image. Consequently, in the present embodiment, it is not necessary for both a color camera and an infrared camera to be prepared.

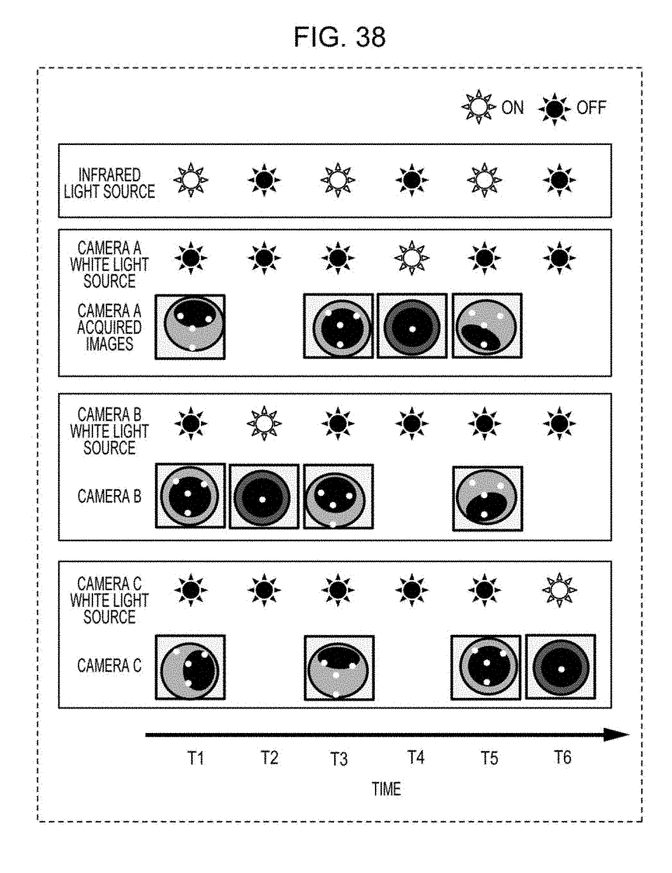

FIG. 38 is a drawing describing a principle whereby color imaging is performed at a timing at which the line of sight of an eyeball of a cow is directly facing an imaging optical axis. The plurality of white light source-equipped color cameras 1040 in the imaging dome are taken here as camera A, camera B, and camera C. These are cameras belonging to either of the left eye group or the right eye group of FIGS. 32 and 33.

These observe corresponding eyeballs or the cow from different viewpoints. In the period during which the cow is inside the imaging dome, ordinarily a plurality of infrared light sources are lit and the white light source-equipped color cameras 1040 function as infrared monochrome cameras and are continuously tracking the line of sight of an eyeball of the cow. At time T1 for example, the infrared light sources are on and cameras A, B, and C have acquired images of the line of sight of the eyeball. At such time, at camera B, it is determined that the line of sight is directly facing, and therefore, at the next instant, time T2, the infrared light sources turn off, the white light source of camera B simultaneously turns on, and a color image of the pupil is captured by camera B. Once again, from the next instant, the infrared light sources turn on and tracking of the line of sight of the eyeball is restarted. Then, at time T3, it is determined that the line of sight is directly facing at camera A, and therefore, at the next instant, time T4, the infrared light sources turn off, the white light source of camera A simultaneously turns on, and a color image of the pupil is captured by camera A.

Next, mutual control of the right eye group and left eye group will be described. In the present embodiment, it is necessary for pupil images of both eyes of one individual cow to be captured by means of coaxial illumination. Therefore, from among the cameras belonging to the group 2010 for the left eye or the group 2020 for the right eye of the cow, one camera ordinarily emits light. However, the white light source momentarily emits light brightly in order to capture images of the respective eyes, and therefore it is also feasible for the cow to be initially startled by the light emitted from either the left or right side and in the next instant to run away from the imaging dome, and in this case, the chance to capture an image of the other eye is lost. In order to avoid this, it is desirable that the left and right groups capture images with the white light sources emitting light at the same time.

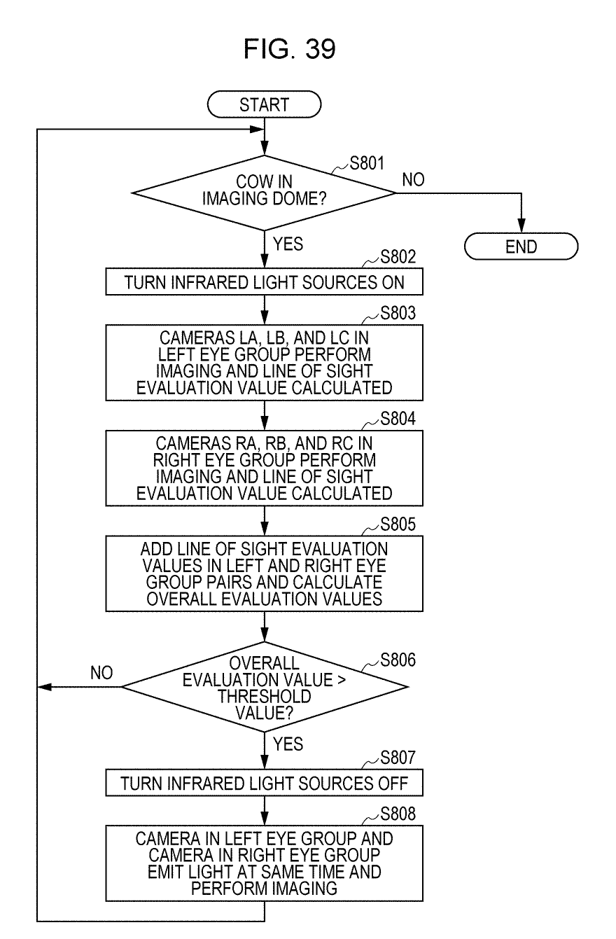

FIG. 39 is a flowchart describing a fixed algorithm for detecting the optimum timing therefor, and imaging performed by the illumination light sources and the cameras is all controlled by the control unit 1030 in accordance with this flowchart. In S801, it is determined whether or not the cow is present inside the imaging dome, and processing ends if not present. This determination is carried out by the pressure sensor 1060 or the like in FIGS. 31A and 31B. When the cow is present inside the imaging dome, in step S802, the infrared light sources turn on and eyeball tracking starts. In the next steps S803 and S804, each of the plurality of viewpoint cameras of the left eye group and the right eye group independently acquire infrared images and determine the line of sight of the eyeballs by means of image processing. The degree to which the line of sight is directly facing a camera optical axis is calculated and taken as a line of sight evaluation value. Then, in step S805, an overall evaluation value is calculated with respective line of sight evaluation values being calculated for each pair of one camera belonging to the left eye group and one camera belonging to the right eye group. Then, in the case where an overall evaluation value has exceeded a threshold value in step S806, the infrared light sources are turned off in step S807, and then in step S808 the white light sources of the corresponding pair of a camera of the left eye group and a camera of the right eye group emit light at the same time to capture respective color images.

Hereinabove, the line of sight of an eyeball and the optical axes for illumination and imaging match. However, a color irregularity is nevertheless present within the captured pupil. This is because reflected color from the tapetum region of the retina is a green to blue color but becomes what is known as “red eye” in the non-tapetum region due to blood vessels being captured, and these two types of reflected light are mixed. In the case of a retina image, the tapetum region and the non-tapetum region are clearly distinct as regions, but in the case of a pupil image, the reflected light from both regions is in a defocused state and an image is produced in which the regions are separated in an indistinct manner.

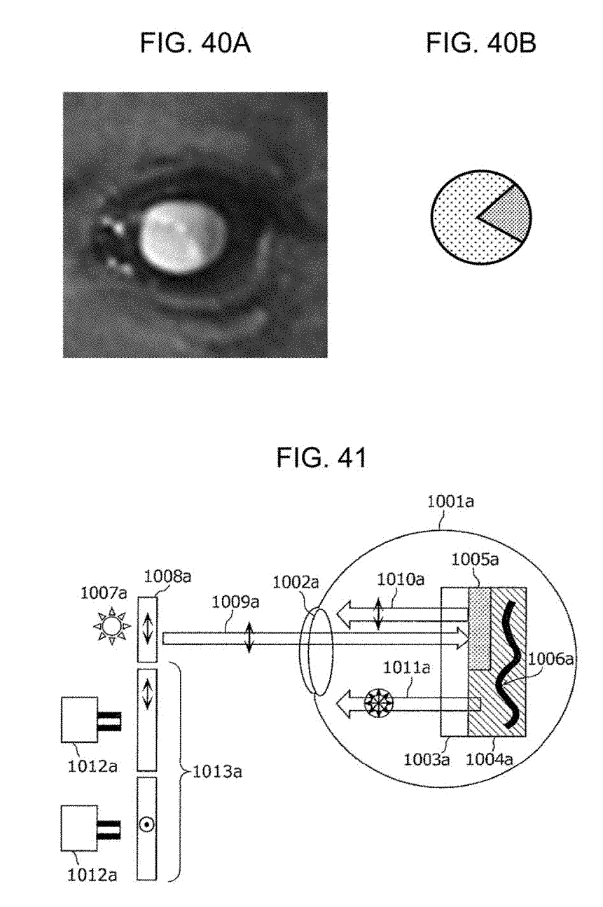

FIGS. 40A and 40B are pupil images of an eyeball of a cow, in which the wide regions on the left side of the pupil images are tapetum regions having a blue-green color, and on the right side are non-tapetum regions having a red-brown color. In the present embodiment, it is important to acquire reflected light from the tapetum region, and reflected light from the non-tapetum region is noise and therefore may be eliminated. However, it has been established that the spectrum of the blue-green color from the tapetum region is not actually within the short wavelength of the blue color and is distributed across a wide wavelength band. For example, in Shuqing HAN, Naoshi KONDO, Yuichi OGAWA, Shoichi MANO, Yoshie TAKAO, Shinya TANIGAWA, Moriyuki FUKUSHIMA, Osamu WATANABE, Namiko KOHAMA, Hyeon Tae KIM, Tateshi FUJIURA, “Estimation of Serum Vitamin A Level by Color Change of Pupil in Japanese Black Cattle”, the red component of a color camera is used to estimate the vitamin A blood concentration from the tapetum colors. However, this is considered to be evidence that the tapetum colors have characteristic reflection characteristics in a central wavelength of 600 nm to 650 nm which is a wavelength band having typical red spectral characteristics. Consequently, it is difficult to separate the tapetum colors using a color filter. For example, when blue of 600 nm or less to the vicinity of yellow is assumed to be the tapetum colors and are separated and determined, the spectral features that are important for estimating the vitamin A blood concentration are discarded.

Thus, in the present embodiment, polarization characteristics are used to separate the tapetum colors.

FIG. 41 is a drawing depicting a principle for separating the aforementioned two regions. 1001a indicates an eyeball cross section, and a pupil 1002a constitutes an opening. 1003a to 1005a schematically indicate a cross-sectional view of a retina, a retina 1003a is a transparent body, a tapetum region 1005a having a blue-green color and high reflectance is present behind a portion of the retina, and a non-tapetum region is constituted by a black choroid 1004a in which blood vessels are abundantly present. When a white light source 1007a transmits through a linear polarizing plate 1008a and illuminates the pupil in a structure such as this, linear polarized illumination 1009a transmits through the pupil 1002a, enters inside the eyeball, reaches at least the retina 1003a which is a transparent body, and reflects. At such time, reflected light 1010a from the tapetum region maintains linear polarization due to the tapetum having the properties of a mirror surface. However, with regard to reflected light 1011a from the non-tapetum region, light reaches the deep section of the choroid and then scatters due to strong forward scattering and returns, and during this process the polarization is lost. These instances of reflected light are observed in a focused state in the pupil 1002a by external color cameras 1012a. Here, to illustrate the principle, FIG. 41 is drawn in such a way that camera-side linear polarizing plates 1013a are installed in front the lenses of the external color cameras 1012a, and the illumination-side polarizing plate 1008a is rotated, the angle thereof is adjusted, and two polarized images of a parallel state and an orthogonal state are acquired. However, in practice, the color polarization camera 4020 is able to simultaneously capture and output two color images having polarization axes that are parallel and orthogonal with respect to the polarization axis of the polarized illumination, and it is therefore possible for this processing to be carried out simultaneously.

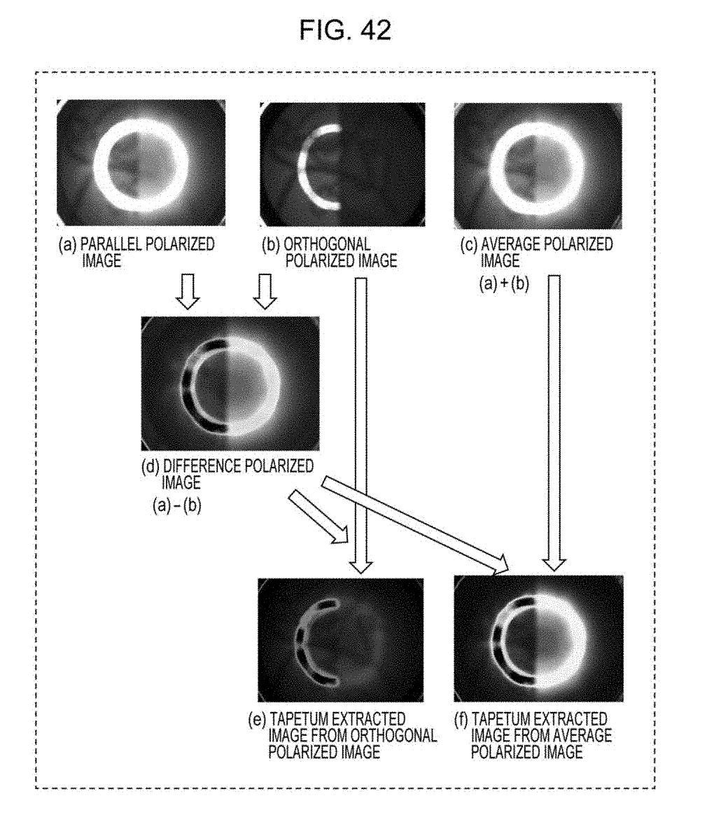

FIG. 42 is a drawing depicting an experiment for separating the tapetum region using a simulated retina model, in which a transparent sheet having a blood vessel pattern is placed on a total diffusion plate to simulate the choroid, a blue sheet is placed thereon in the right-half region to simulate the tapetum, and a transparent acrylic sheet is placed thereon to simulate the retina. White ring illumination having linear polarization is radiated from directly above this simulated retina to acquire (a) a parallel polarized image and (b) an orthogonal polarized image. These two images are equivalent to images captured by the two different imaging elements 4080 and 4060 in FIG. 34.

In (b), a non-polarized reflection image is brightly captured with polarized light that returns from the pseudo non-tapetum region in the left half having become disarranged, and mirror surface reflection light from the pseudo tapetum region in the right half maintains those polarization characteristics and as is therefore blocked. These are added and averaged ((a)+(b)) to obtain (c) an averaged polarized image. This (c) is an image that is close to the capturing of an ordinary color image, in which an image from the pseudo tapetum in the right half and an image from the pseudo non-tapetum region in the left half are both brightly captured, and therefore equates to being captured as a pupil image in which the tapetum region and the non-tapetum region are uneven.

Next, the difference ((a)−(b)) between the (a) parallel polarized image and the (b) orthogonal polarized image is acquired to obtain (d) a difference polarized image. In the difference polarized image, reflected light from the tapetum region is extracted. Thus, using this image, for example, a (e) tapetum extracted image from the orthogonal polarized image is obtained when (d) and the (b) orthogonal polarized image are multiplied, and a (f) tapetum extracted image from the averaged polarized image is obtained when (d) and the (c) averaged polarized image are multiplied. Therein, a color specific to the blue-green tapetum region in the right half is extracted, the left half become a black background and the remainder of the reflection of the ring illumination, and therefore, when image averaging is carried out, the reflected color of the tapetum region is obtained as the main component.

### Embodiment 8

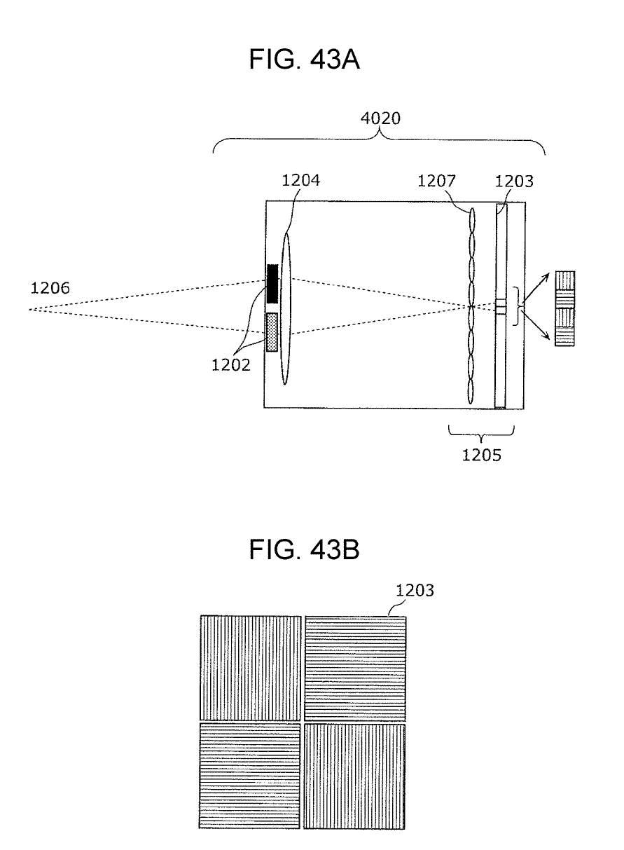

FIG. 43A is a drawing depicting a polarization imaging device of embodiment 8, and a difference from embodiment 7 is the configuration of the color polarization camera 4020.

In the present embodiment, color separation for the RGB wavelength band is executed by means of color filters 1202 arranged on an opening of an objective lens 1204. In the camera 4020 depicted, color separation and polarization imaging are carried out using a microlens array-type of color image sensor 1205 in which a microlens array 1207 and a monochrome polarization image sensor 1203 are formed as a single unit.

Return light that has diverged from one point 1206 on the subject transmits though each of the two regions (color filters) 1202 on the objective lens 1204, and reaches the imaging surface of the monochrome polarization image sensor 1203 via the microlens array 1207.

FIG. 43B is a drawing depicting a planar structure of the monochrome polarization image sensor 1203, in which two types of pixel regions having polarization transmission axes of 0° (horizontal) and 90° (vertical) are integrated in a mosaic form.

In this case, light rays that pass through the two regions 1202 on the objective lens 1204 reach different pixels. Therefore, an image formed on the monochrome polarization image sensor 1203 is in its entirety an image of the subject; however, in detail, color images from the two different regions 1202 are encoded. By carrying out digital image processing for selecting and integrating pixels, color images can be generated with the images transmitted through the two regions 1202 being separated.

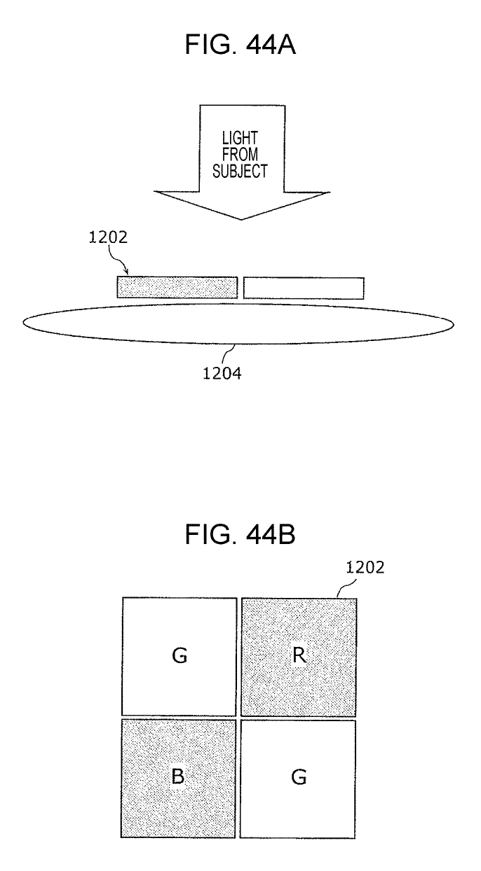

FIG. 44A is a drawing depicting a cross-sectional structure of the objective lens 1204 and the color filter regions (color filters) 1202. As depicted in FIG. 44B, on the objective lens that constitutes an opening, three different types of color filters R, G, B, and G are arranged in two rows by two columns. It should be noted that the installation order of the color filter regions 1202 and the objective lens 1204 may be the reverse of that in FIG. 44A for light from the subject.

The arrangement method for the color filter regions 1202 may be different from that in FIG. 44B. The color filter regions 1202 can be formed from an organic substance, a photonic crystal, or any other filter material. A filter that exhibits the spectral characteristics depicted in FIG. 37B may be used for an RGB color filter.

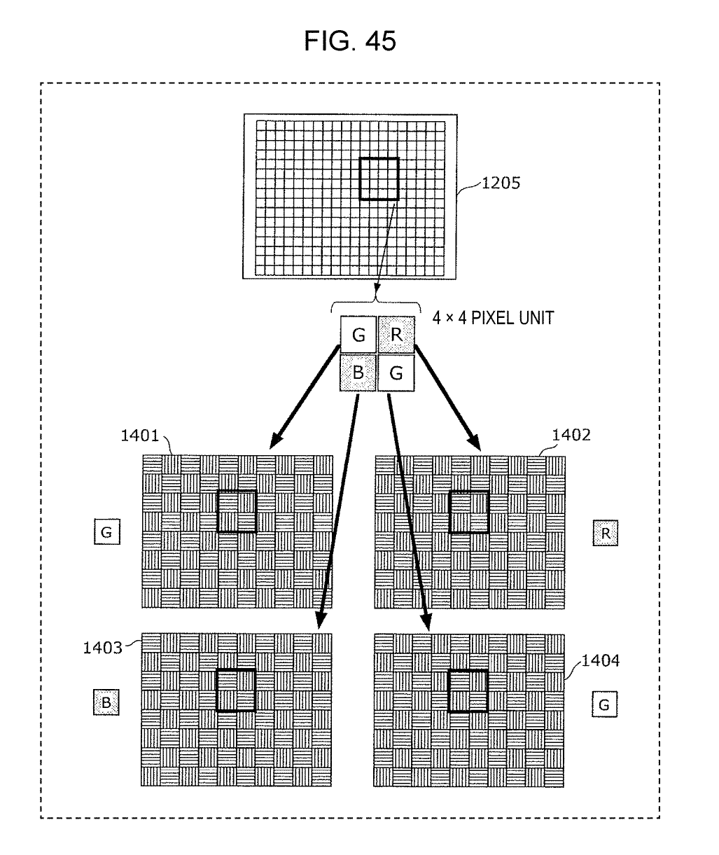

FIG. 45 is a drawing describing processing for pixel selection and reintegration in which a color polarized image is generated from imaging results obtained using the microlens array-type of color image sensor 1205. A pixel unit arranged over four rows by four columns on an image on the microlens array-type of color image sensor 1205 corresponds as a pixel for a light ray that has transmitted through a filter region of a 4×4 region in the opening of the objective lens. Four two-row by two-column pixels in the top left, top right, bottom left, and bottom right are selected and reintegrated across the entire image. The resolution drops to ¼×¼ due to this processing; however, it is possible to separate a polarized mosaic image 1401 for G, a polarized mosaic image 1402 for R, a polarized mosaic image 1403 for B, and a polarized mosaic image 1404 for G.

In the present embodiment also, polarized images produced by light polarized in the polarization transmission axis directions of 0° and 90° in each wavelength band of R, G, and B can be obtained at the same time, and therefore polarized image processing that is similar to that of embodiment 7 becomes possible.

In the present embodiment, as depicted in FIGS. 43A and 43B, an image sensor in which two types of mosaic polarizers of 0° (horizontal) and 90° (vertical) are aligned is given as an example of a monochrome polarization image sensor; however, it should be noted that four types of mosaic polarizers such as 0°, 45°, 90°, and 135° may be aligned therein, for example. If there are three or more types of polarizers, polarized illumination does not have to be used, and it becomes possible to calculate the principal axis and degree of polarization of the polarization of any incident light in a general scene. Thus, in the case where the illumination light source is turned on, water droplet detection processing or the like on the imaging dome 1020 is realized using the pixels of 0° and 90° from among 0°, 45°, 90°, and 135°, and in the case where the illumination light source is turned off, by using all four types of polarizers, it becomes possible to operate as a polarization camera capable of imaging in which the eyeball surface state of the cow is detected and reflection from the curved imaging dome 1020 is eliminated.

### Embodiment 9

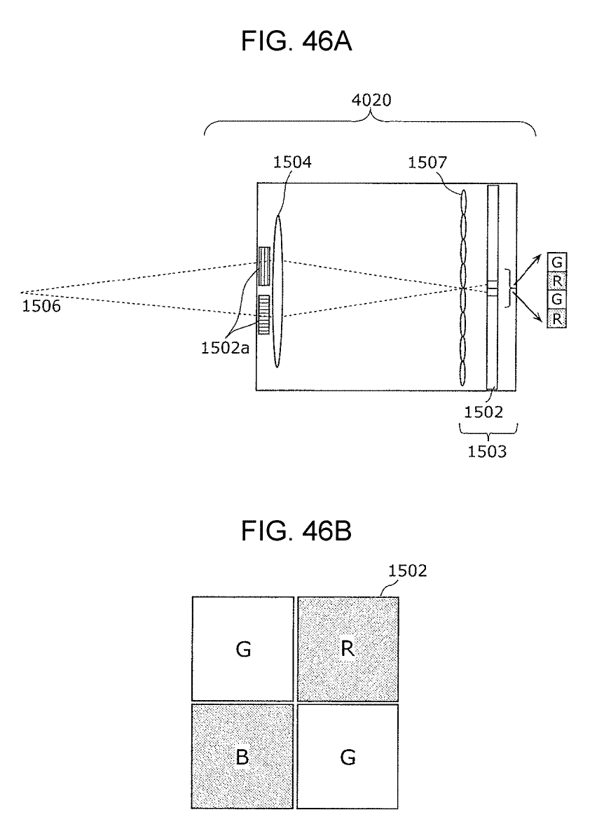

FIG. 46A is a drawing depicting a polarization imaging device of embodiment 9, and a difference from embodiment 7 is the configuration of the color polarization camera 4020.

In the present embodiment, the separation of light in the polarization transmission axis directions of 0° and 90° is executed by means of polarizing mosaic filters 1502a arranged on an opening of an objective lens 1504.

In the camera 4020 depicted, color separation and polarization imaging are carried out using a microlens array-type of color image sensor 1503 in which a microlens array 1507 and a single-plate color imaging element 1502 having wavelength band pixels for R, G, and B are formed as a single unit. Return light that has diverged from one point 1506 on the subject transmits though each of two regions 1502a on the objective lens 1504, and reaches the single-plate color imaging element 1502 (1503), in which a color mosaic is arranged, via the microlens array 1507. Pixels are reached having different configurations from having passed through the two regions (polarizing mosaic filters) 1502a on the objective lens 1504. Therefore, an image formed on the single-plate color imaging element 1502 (1503) is in its entirety an image of the subject but in detail becomes an image formed from images of different polarization regions of 0° and 90°. Each region corresponds to color mosaic 2×2 pixels on the color imaging element.

FIG. 46B is a drawing depicting a planar structure of the single-plate color imaging element 1502, which may be an ordinary single-plate color imaging element in which Bayer mosaic types of RGB pixel regions are integrated. A filter that exhibits the spectral characteristics depicted in FIG. 37B may be used for an RGB color filter.

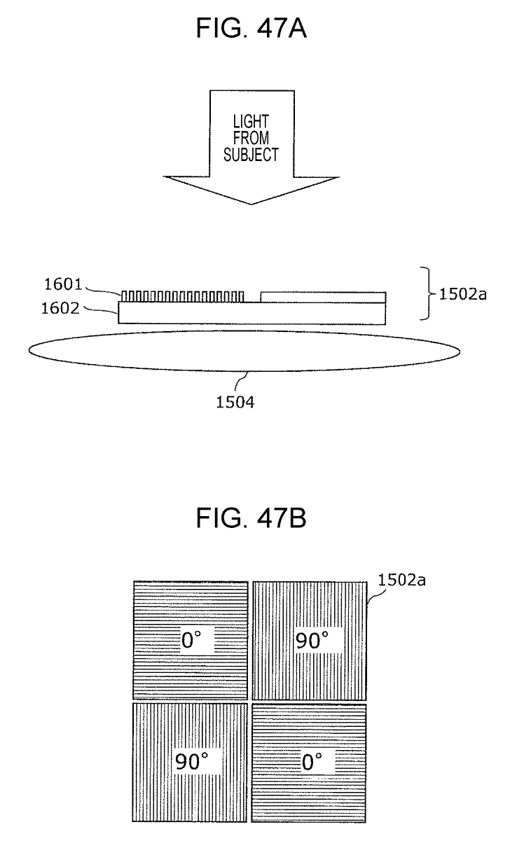

FIG. 47A is a drawing depicting a cross-sectional structure of the polarizing filter regions (polarizing mosaic filters) 1502a of the opening in the present embodiment. In this example, a metal wire grid layer is used as the polarizing filters. A wire grid layer 1601 has metal wires having pitches of approximately 100 nm formed on a transparent substrate 1602, and is capable of realizing a polarizing operation over a wide band in the visible light to infrared range.

The objective lens 1504 is installed at the stage subsequent to the polarizing filter regions 1502a. The arrangement order of the wire grid layer 1601 and the objective lens 1504 and whether or not there is a gap between the wire grid layer 1601 and the objective lens 1504 are design matters. The polarizing plate, if realizing a polarizing operation over a wide band within the visible light range, is not restricted to a wire grid layer, and a polymer polarizing plate or the like can also be used. A wire grid layer can be formed from a variety of metal materials such as aluminum (Al). The wire grid layer 1601 is not restricted to having a single-layer structure, and may have a multilayer structure. In such cases, a light absorption layer may be arranged on the outermost surface layer to suppress reflection. Gaps in stacked wire grids may be filled with another material to enhance mechanical strength. A coating may be applied in order to protect the surface of the wire grid from chemical reactions.

FIG. 47B is a drawing depicting a planar structure of the polarizing filter regions 1502a. These polarizing filter regions 1502a are configured of a 2×2 total of four polarizing filters having polarization transmission axes of 0° and 90°.

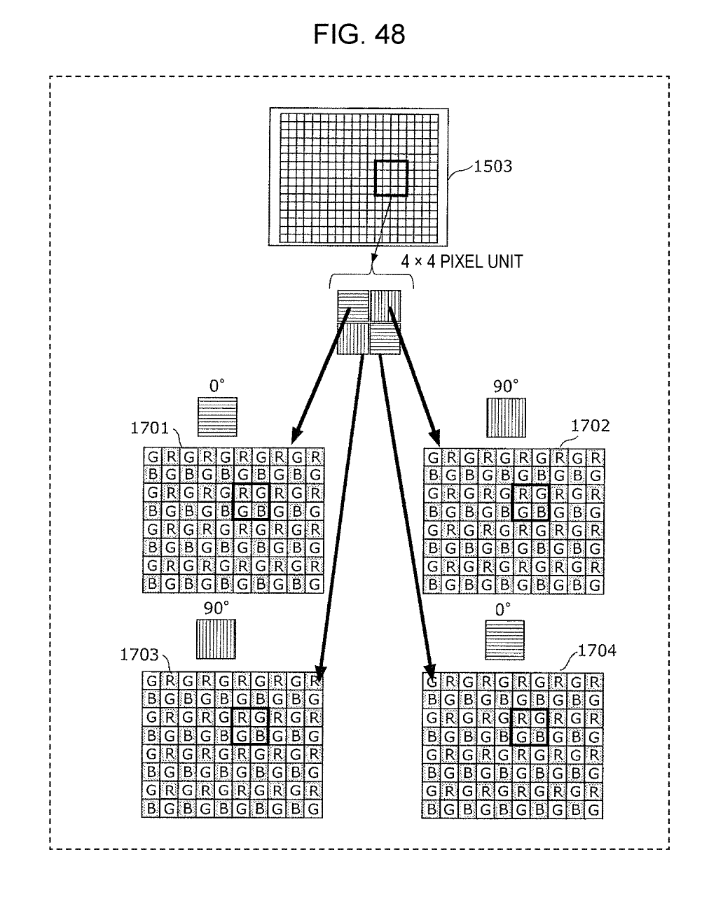

FIG. 48 is a drawing describing pixel selection and reintegration processing in which a color polarized image is generated from imaging results obtained using the microlens array-type of color image sensor 1503. The 4×4 pixel unit on an image on the sensor 1503 corresponds to a light ray from the filters of the four regions in the objective lens opening, and therefore, by selecting and reintegrating 2×2 pixels in the top left, top right, bottom left, and bottom right across the entire image, the resolution drops to ¼×¼, but it is possible to separate the color mosaic images 1701 and 1704 for R, G, B, and G corresponding to the polarization transmission axis of 0° and the color mosaic images 1702 and 1703 for R, G, B, and G corresponding to the polarization transmission axis of 90°. Full color and infrared polarized images of 0° and 90° can be obtained therefrom by carrying out publicly-known color mosaic interpolation processing.

A benefit of the present embodiment is that, because it is possible to install polarizing plates in the lens opening, the sizes of the individual polarizing mosaic elements can be made to be larger than when arranged on an imaging element. For example, in a polarizing mosaic type of imaging element used in the other aforementioned embodiments, the length of the metal wire that forms polarizing mosaic units is equal to the pixel size of the imaging element and is typically 1 to 3 μm. With such a minute size, the length of wire grid and the number of repetitions are limited even if the pitches between the individual metal wires of the wire grid are minute. As a result, the extinction ratio performance as a polarizing plate drops to approximately 10:1. In the present embodiment, a comparatively large wire grid polarizing plate in which the size of the lens opening is approximately 0.5 mm=500 μm can be used, and a high extinction ratio of approximately 100:1 can be realized, which is extremely advantageous in terms of performance.

### Embodiment 10

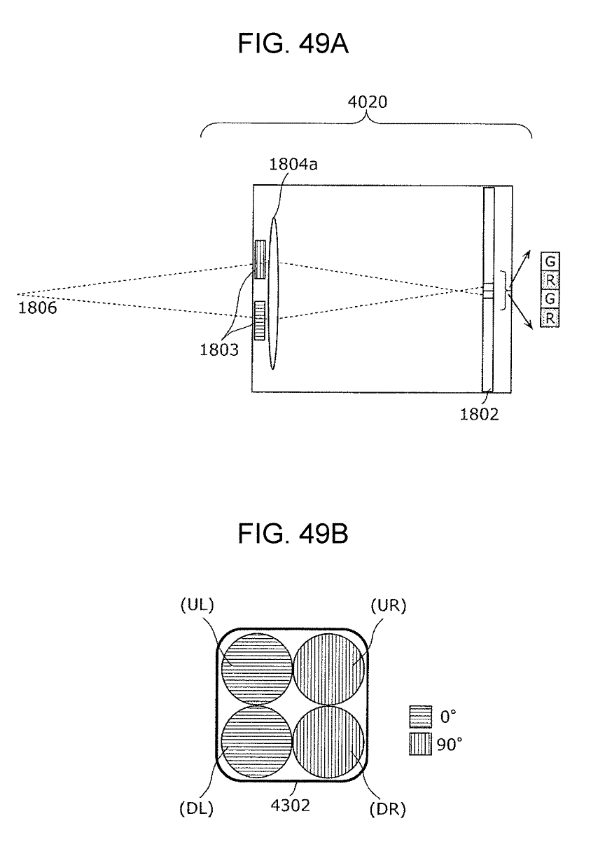

FIG. 49A is a drawing depicting a polarization imaging device of the present embodiment, and a difference from embodiment 7 is the configuration of the color polarization camera 4020.

In the present embodiment, the separation of light in the polarization transmission axis directions of 0° and 90° is executed by means of polarizing mosaic filters 1803 arranged on each opening of a plurality of objective lenses 1804a. This multi-lens color camera has a color imaging element 1802 that has three wavelength band pixels for R, G, and B on an imaging surface. The configuration of this color imaging element is that of an ordinary single-plate color image sensor and is therefore omitted; however, an RGB color filter may have the spectral characteristics depicted in FIG. 37B.

Return light that has diverged from one point 1806 on the subject transmits though the polarizing filter regions (polarizing mosaic filters) 1803 on the 2×2 total of four multi-objective lenses 1804a and reaches the color imaging element 1802 in which a color mosaic is arranged. The images of each region on the objective lens become different images juxtaposed on the imaging surface.

FIG. 49B is a drawing depicting polarization axes of polarizing filters corresponding to openings (UL), (UR), (DL), and (DR) of the aforementioned four multi-objective lenses, having installed therein polarizing plates in which (UL) and (DL) are 0° and (UR) and (DR) are 90°.

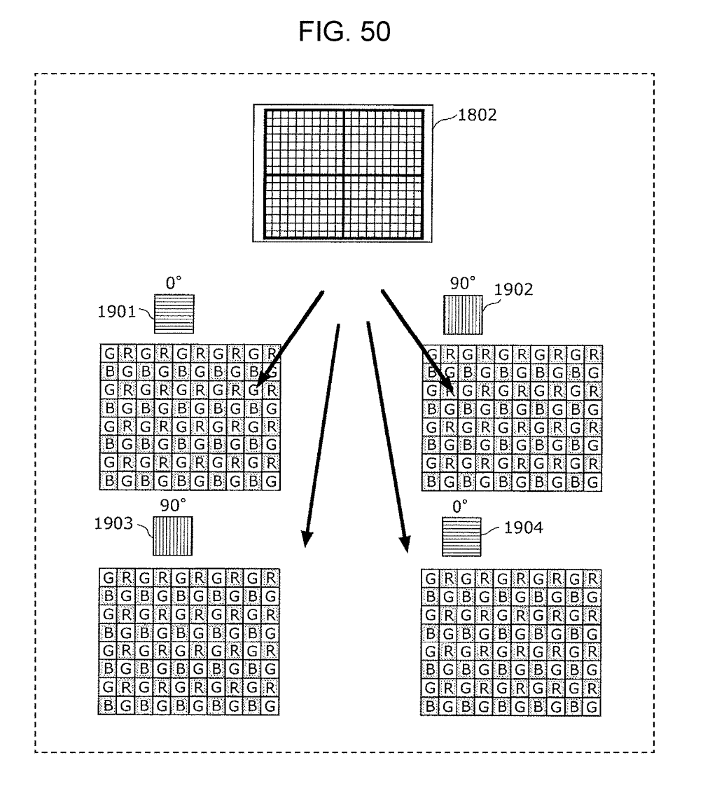

FIG. 50 is a drawing describing pixel selection processing in which a polarized image is generated from imaging results obtained by using a multi-lens color camera. Images having transmitted through the four regions in the four objective lens openings on an image on the color imaging element 1802 are juxtaposed in the top left, top right, bottom left, and bottom right. Thus, when the acquired images are isolated and separated, the resolution drops to ¼×¼, but it is possible to separate the color mosaic images 1901 and 1904 for R, G, and B corresponding to the polarization transmission axis of 0° and the color mosaic images 1902 and 1903 for R, G, and B corresponding to the polarization transmission axis of 90°. Full color and infrared polarized images of 0° and 90° can be obtained therefrom by carrying out publicly-known color mosaic interpolation processing.

According to the present embodiment, a polarizing plate is installed in the lens opening, and therefore the sizes of the individual polarizing mosaic elements can be made to be larger than when installed on an imaging element.

Hereinabove, a camera system, a feeding system, and an imaging method according to one or more aspects have been described on the basis of the aforementioned embodiments; however, the present disclosure is not limited to the aforementioned embodiments. Modes in which various modifications conceived by a person skilled in the art have been implemented in the present embodiments, and modes constructed by combining the constituent elements in different embodiments may also be included within the scope of the present disclosure provided they do not depart from the purpose of the present disclosure.

It should be noted that, in the aforementioned embodiments, the constituent elements may be configured by using dedicated hardware, or may be realized by executing a software program suitable for the constituent elements. The constituent elements may be realized by a program execution unit such as a CPU or a processor reading out and executing a software program recorded in a recording medium such as a hard disk or a semiconductor memory. Here, software that realizes the camera system or the feeding system of the aforementioned embodiments is a computer program that causes a computer to execute each step indicated in the flowcharts of any of FIGS. 3, 21, 24 to 26C, and 39.

Furthermore, in the present disclosure, all or some of the units and devices, or all or some of the functional blocks of the block diagrams depicted in FIGS. 1, 4, 15, 23, 29, 30, 31A, and 31B may be executed by one or more electronic circuits including a semiconductor device, a semiconductor integrated circuit (IC), or a large-scale integration (LSI). An LSI or an IC may be integrated in one chip or may be configured by combining a plurality of chips. For example, function blocks other than storage elements may be integrated in one chip. Here, reference has been made to an LSI or an IC; however, the name that is used is different depending on the degree of integration and these may be referred to as a system LSI, a very-large-scale integration (VLSI), or an ultra-large-scale integration (ULSI). A field-programmable gate array (FPGA) that is programmed after the manufacture of an LSI, or a reconfigurable logic device with which connection relationships inside an LSI can be reconfigured or circuit segments inside an LSI can be set up is also able to be used for the same purpose.

In addition, it is possible for all or some of the functions or operations of the units, devices, or some of the devices to be executed by software processing. In this case, software is recorded in a non-transitory recording medium such as one or more ROMs, optical discs, or hard disk drives, and in the case where the software is executed by a processing device (processor), the software causes specific functions within the software to be executed by the processing device (processor) and peripheral devices. The system or the device may be provided with one or more non-transitory recording mediums on which the software is recorded, the processing device (processor), and required hardware devices such as an interface.

The present disclosure can be applied to a camera system that is set up in a cattle barn, for example, and captures images of an eyeball of a cow or the like. In this camera system, individual authentication and a lesion diagnosis can both be carried out, and a lesion diagnosis or a vitamin A blood concentration can be estimated, in a non-contact manner, in other words, without causing unnecessary stress to an animal such as a cow. Furthermore, the camera system has the effect of it being possible to acquire a reflected color from the retina tapetum region in a stable manner and with good accuracy. Furthermore, it thereby becomes possible to accurately estimate the vitamin A blood concentration of beef cattle. Furthermore, the present disclosure is effective not only for cows but also for pet animals such as dogs or cats that have a tapetum layer, and can be used also as an ophthalmologic diagnosis device in a veterinary clinic.

What is claimed is:

1. A camera system that captures images of eyeballs of an animal, comprising:
a first illumination device that illuminates an eyeball of the animal;
a fundus imaging camera that captures a fundus image of the eyeball illuminated by the first illumination device;
a second illumination device that illuminates an eyeball of the animal at the same timing as the first illumination device;
a pupil imaging camera that captures a pupil image of the eyeball illuminated by the second illumination device; and
an output circuit that outputs the fundus image as identification information of the animal, and outputs the pupil image as biological information of the animal corresponding to the identification information.

2. The camera system according to claim 1,
wherein the first illumination device is an infrared illumination device or a white illumination device, and
the second illumination device is a white illumination device.

3. The camera system according to claim 1, further comprising:
an infrared illumination device; and
a line of sight detector that detects a line of sight of the animal,
the fundus imaging camera capturing a fundus image for detecting the line of sight of the eyeball illuminated by the infrared illumination device,
the line of sight detector detecting the line of sight of the animal using the fundus image for detecting the line of sight,
the first illumination device and the second illumination device illuminating the eyeballs, based on the detected line of sight of the animal, and
the fundus imaging camera capturing the fundus image of the eyeball, and the pupil imaging camera capturing the pupil image of the eyeball.

4. The camera system according to claim 3,
wherein the first illumination device and the second illumination device illuminate the eyeballs when the detected line of sight of the animal is the same as an imaging optical axis of the fundus imaging camera.

5. The camera system according to claim 1,
wherein the second illumination device emits light within 0.3 sec from a point in time at which the first illumination device emitted light.

6. The camera system according to claim 1, further comprising:
a measurer that measures a pupil constriction velocity of the animal,
the second illumination device once again illuminating the eyeball of the animal, within 0.3 sec from a point in time of having emitted light at the same timing as the first illumination device,
the pupil imaging camera capturing a plurality of pupil images in accordance with illumination performed by the second illumination device, and
the measurer measuring the pupil constriction velocity of the animal using the plurality of pupil images.

7. The camera system according to claim 1,
wherein, when an angle formed by an illumination optical axis of the first illumination device and an imaging optical axis of the fundus imaging camera is θ1, and
an angle formed by an illumination optical axis of the second illumination device and an imaging optical axis of the pupil imaging camera is θ2,
a condition θ1≦θ2 is satisfied.

8. The camera system according to claim 1,
wherein the fundus imaging camera has a first objective lens,
the pupil imaging camera has a second objective lens, and,
when a distance between the first objective lens and a position of a surface of an eyeball of the animal is L1, and
a distance between the second objective lens and a position of a surface of an eyeball of the animal is L2,
a condition L1<L2 is satisfied.

9. The camera system according to claim 1, further comprising:
an identifier that identifies the individual animal using the fundus image,
the animal not being illuminated by the second illumination device when the identifier is not able to identify the individual animal.

10. The camera system according to claim 1, further comprising:
a determiner that determines whether or not the fundus image includes a lesion,
the animal not being illuminated by the second illumination device when the fundus image includes the lesion.

11. The camera system according to claim 9, further comprising:
cover glass that covers the fundus imaging camera, between the fundus imaging camera and the animal; and
a cover glass cleaning device that cleans the cover glass when a number of times the identifier has not been able to identify the individual animal is equal to or greater than a predetermined number of times.

12. A feeding system that feeds an animal using a fundus image and a pupil image of the animal captured by a camera system, the camera system being provided with:
a first illumination device that illuminates an eyeball of the animal;
a fundus imaging camera that captures the fundus image of the eyeball illuminated by the first illumination device;
a second illumination device that illuminates an eyeball of the animal at the same timing as the first illumination device;
a pupil imaging camera that captures the pupil image of the eyeball illuminated by the second illumination device;
an output circuit that outputs the fundus image as identification information of the animal, and outputs the pupil image as biological information of the animal corresponding to the identification information;
an estimator that estimates a concentration of vitamin A in blood of the animal using the pupil image; and
an interface that outputs a signal for switching a composition of feed, corresponding to the concentration of the vitamin A estimated by the estimator.

13. An imaging method for capturing images of eyeballs of an animal, including:
illuminating an eyeball of the animal using a first illumination device;
capturing a fundus image of the eyeball illuminated by the first illumination device, using a fundus imaging camera;
illuminating an eyeball of the animal at the same timing as the first illumination device, using a second illumination device;
capturing a pupil image of the eyeball illuminated by the second illumination device, using a pupil imaging camera; and
outputting the fundus image as identification information of the animal, and outputting the pupil image as biological information of the animal corresponding to the identification information, using an output circuit.

14. An imaging device, including:
a first camera that captures a first image of a first eye illuminated by infrared light radiated from an infrared light radiator, an animal having the first eye and a second eye that is different from the first eye;
a second camera, a distance between an objective lens of the first camera and the first eye being less than a distance between an objective lens of the second camera and the second eye;
a decider that decides which one of processes including a first process and a second process is to be executed, each of the processes, when executed, being executed after the first image is captured; and
an outputter that outputs a plurality of images in the second process,
in the first process, the first camera capturing an additional first image of the first eye illuminated by additional infrared light radiated from the infrared light radiator,
in the second process, (i) the first camera capturing a second image of the first eye illuminated by first white light radiated from a first white light radiator, (ii) the second camera capturing a third image of the second eye illuminated by second white light radiated from a second white light radiator, and (iii) the second camera capturing a fourth image of the second eye illuminated by the second white light,
the plurality of images including the second image, the third image, and the fourth image, and
a time interval between the first image being captured and the additional first image being captured being greater than a time interval between the third image being captured and the fourth image being captured.

15. The imaging device according to claim 14, further including:
a decider that decides the one process, based on luminance data of a pixel of the first image.
